(19) United States
(12) Patent Application Publication
(10) Pub. No.: US 2023/0230697 A1
(43) Pub. Date: Jul. 20, 2023

(54) SYSTEMS AND METHODS FOR EVALUATING A SUBJECT FOR INFLAMMATORY BOWEL DISEASE MANAGEMENT

(71) Applicant: Icahn School of Medicine at Mount Sinai, New York, NY (US)

(72) Inventors: Marla C. Dubinsky, New York, NY (US); Laurie Keefer, New York, NY (US)

(21) Appl. No.: 18/002,075

(22) PCT Filed: May 26, 2021
(86) PCT No.: PCT/US21/34333

§ 371 Date: Dec. 16, 2022

Related U.S. Application Data
(60) Provisional application No. 63/045,714, filed on Jun. 29, 2020.

(51) Int. Cl. G16H 50/20

(52) U.S. Cl. CPC G16H 50/20

(57) ABSTRACT

Systems and methods are provided for evaluating a subject for inflammatory bowel disease (IBD) care coordination strategy. A resilience score is determined based on a plurality of different resilience domain assessments of the subject. When the resilience score satisfies a care coordination threshold, the subject is enlisted in an IBD care coordination program. An IBD severity score for the subject is determined based, at least in part, on one or more risk factors. Both the resilience score and the IBD severity score are used to assign the subject to a category in a plurality of categories. At least one time-limited care plan is determined at a first time for the subject based upon the identity of the assigned category. The at least one time-limited care plan is prioritized by an outcome of one or more resilience domain assessments in the plurality of different resilience domain assessments.

### CROSS REFERENCE TO RELATED APPLICATIONS

This application claims priority to U.S. Provisional Patent Application No. 63/045,714, filed Jun. 29, 2020, the content of which is hereby incorporated by reference, in its entirety, for all purposes.

### TECHNICAL FIELD

The present disclosure relates generally to systems and methods for evaluating a subject for inflammatory bowel disease (IBD) management (e.g., providing a coordinated care plan to the subject).

### BACKGROUND

Approximately 1.6 million Americans of all ages are affected by inflammatory bowel disease (e.g., IBD), and up to 70,000 new cases are diagnosed every year. See e.g., Crohn's & Colitis Foundation/United Health Data 2019; and Iskandar et al. (2012) “Biomarkers in Inflammatory Bowel Disease: Current Practices and Recent Advances” Transl Res 159(4), 313-325. In addition, IBD is one of the most expensive chronic medical conditions to treat, with medical costs of up to $40,000 per year per patient, and an estimated indirect cost per year in excess of $5 billion in terms of decreased workplace productivity. See e.g., Crohn's & Colitis Foundation/United Health Data 2019; and Park et al. (2011) “Inflammatory Bowel Disease-Attributable Costs and Cost-effective Strategies in the United States: A Review” Inflamm Bowel Dis 17(7), 1603-1609. Beyond the financial cost, IBD can be a devastating disease, and patients need significant support from the health care system to manage their illness.

However, current treatment regimens fail patients at multiple points. First, children, who make up about 25% of the patients diagnosed each year with IBD, have disproportionately poorer patient outcomes due in part to the difficulty in smoothly transitioning from pediatric to adult health care providers. See Goodhand et al. (2011) “Adolescents with IBD: The importance of structured transition care” J. Crohn's and Colitis 5, 509-519; and Carlsen et al. (2017) “Self-efficacy and Resilience Are Useful Predictors of Transition Readiness Scores in Adolescents with Inflammatory Bowel Diseases” Inflamm Bowel Dis 23(3), 341-346. In addition, IBD patients frequently initiate changes in their specialist health care provider (often due to negative interactions). See van Langenberg et al. (2012) “Satisfaction with patient-doctor relationships in inflammatory bowel diseases: Examining patient-initiated change of specialist” World J Gastroenterol 18(18), 2212-2218. Delays or interruptions in treatment increase the risk of patients developing complications requiring hospitalization; mental health concerns due to unnecessary suffering are also significantly increased. See Nowakowski et al. (2016) “Psychiatric illnesses in inflammatory bowel diseases—psychiatric comorbidity and biological underpinnings” Psychiatr. Pol. 50(6): 1157-1166. Hospitalizations are expensive for the medical system and unpleasant for patients. Park et al. (2011) “Inflammatory Bowel Disease-Attributable Costs and Cost-effective Strategies in the United States: A Review” Inflamm Bowel Dis 17(7), 1603-1609.

Moreover, a review of Mount Sinai IBD population health data indicates that 46 percent of IBD patients drive approximately ninety percent of the total cost of treating such patients.

There is thus a clear need for improved long-term management of IBD patients.

Given the structural issues in the medical system and the high burden of IBD on patients, additional tools are needed to provide patients with management plans and information for managing their long-term health.

### SUMMARY

The present disclosure addresses the need in the art for systems and methods for evaluating patients with IBD diagnoses for inflammatory bowel disease management (e.g., for determining a coordinated care plan specific to the patient). In particular, the disclosed systems and methods use patient information beyond the initial diagnosis to determine a personalized and coordinated care plan (e.g., determining a disease-management plan specific to the patient). One particular indication of a patient's ability to manage IBD successfully is having high resilience, or the ability to recover from setbacks. See e.g., Carlsen et al. (2017) “Self-efficacy and Resilience Are Useful Predictors of Transition Readiness Scores in Adolescents with Inflammatory Bowel Diseases” Inflamm Bowel Dis 23(3), 341-346. Care plan recommendations can thus be tailored to an individual patient based at least in part on the patient's measured resilience level or by helping the patient increase their resilience. Through the disclosed systems and methods, improved coordinated care plans for the long-term management of IBD for individual patients are provided. Moreover, by customizing care plans to respond to the individualized needs of patients, the disclosed systems and methods lead to improved patient outcome.

One aspect of the present disclosure provides a method of evaluating a subject for an inflammatory bowel disease (IBD) care coordination strategy. The method comprises determining a resilience score, within a range of resilience scores, for a subject, where each of a plurality of different resilience domain assessments of the subject contribute to the resilience score. The method further comprises using the resilience score to determine whether to enlist the subject in an IBD care coordination program. When the resilience score fails to satisfy a care coordination threshold, the subject is not enlisted in the IBD care coordination program. When the resilience score satisfies the care coordination threshold, the method further comprises performing the IBD care coordination program by a procedure comprising determining an IBD severity score, within a range of IBD severity scores, for the subject based, at least in part, on a risk for one or more IBD complications or surgery to alleviate IBD, using both the resilience score and the IBD severity score to assign the subject to a category in a plurality of categories, where each category in the plurality of categories is associated with a unique combination of a sub-range of the range of resilience scores and sub-range of the range of IBD severity scores, determining at least one time-limited care plan, at a first time point, for the subject based upon the identity of the assigned category within the plurality of categories, and prioritizing the at least one time-limited care plan by an outcome of one or more resilience domain assessments in the plurality of different resilience domain assessments.

In some embodiments, the plurality of categories consists of a first category, a second category, a third category, and a fourth category. In some embodiments, the first category comprises a combination of a low resilience score and a high IBD severity score, and is associated with a care plan comprising (i) enrollment in a resilience program, (ii) close disease monitoring of the subject, (iii) tracking one or more of remote symptoms, subject reported outcomes, and biomarkers of the subject associated with IBD, (iv) application of one or more digital behavioral health maintenance and prevention tools associated with IBD, and (v) educational content customized to the subject. In some embodiments, the second category comprises a combination of a high resilience score and a high IBD severity score, and is associated with a care plan comprising (i) periodic resilience score reassessment, (ii) close disease monitoring of the subject, (iii) tracking one or more of remote symptoms, subject reported outcomes, and biomarkers of the subject associated with IBD, (iv) application of one or more digital behavioral health maintenance and prevention tools associated with IBD, and (v) featured educational content. In some embodiments, the third category comprises a combination of a low resilience score and a low IBD severity score, and is associated with a care plan comprising (i) enrollment in a resilience program, (ii) tracking one or more of remote symptoms, subject reported outcomes, and biomarkers of the subject associated with IBD, (iii) application of one or more digital behavioral health maintenance and prevention tools associated with IBD, and (iv) educational content customized to the subject. In some embodiments, the fourth category comprises a combination of a high resilience score and a low IBD severity score and is associated with a care plan comprising (i) periodic resilience score reassessment, (ii) tracking one or more of remote symptoms, subject reported outcomes, and biomarkers of the subject associated with IBD, (iii) application of one or more digital behavioral health maintenance and prevention tools associated with IBD, and (iv) featured educational content.

In some embodiments, the plurality of different resilience domain assessments of the subject comprises two or more, three or more, four or more, or all five of: (i) a general medical factors affecting resilience, (ii) a measure of independence exhibited by the subject, (iii) a nutritional and staminal assessment of the subject, (iv) a psychosocial assessment of the subject, and (v) an assessment of the present ability of the subject to access medical care.

In some embodiments, the plurality of different resilience domain assessments of the subject comprises the subject's experience of physical health, and are one or more of: (i) when the subject is in remission, a report of high impact of symptoms on daily functions, (ii) when the subject has had at least one unplanned hospitalization or emergency department (ED) visit in the previous 12 months, (iii) when the subject has had an IBD-related surgery in the previous 6 months, (iv) when the subject will have surgery in the next 6 months, and (v) a report of chronic pain by the subject.

In some embodiments, the plurality of different resilience domain assessments of the subject comprises the subject's experience of physical health in the form of chronic pain, fatigue, urgency to use the bathroom, incontinence, nausea, and recent or future health care use.

In some embodiments, the plurality of different resilience domain assessments of the subject comprises a measure of independence exhibited by the subject, and the subject is deemed to have inadequate independence such as when the subject is afraid to self-inject or undergo infusion or necessary medical procedures, has a poor relationship with a caregiver or care team, is a child or young adult, has a poor attendance record at work or school, or has frequently cancelled or failed to attend medical appointments.

In some embodiments, the plurality of different resilience domain assessments of the subject comprises a measure of independence exhibited by the subject that indicates that the subject has inadequate independence, and the applying comprises arranging to have the subject meet with a social worker and/or a clinical pharmacist associated with the assigned care plan.

In some embodiments, the at least one time-limited care plan comprises a cognitive behavioral therapy, a low fermentable oligosaccharides, disaccharides, monosaccharides and polyols (FODMAP) diet, or a self-directed hypnosis.

In some embodiments, the determining a resilience score is performed by having the subject complete a questionnaire in an application on a smart phone associated with the subject.

In some embodiments, the procedure further comprises repeating the determining the resilience score after a first predetermined period has elapsed since the first time point, where: when the resilience score fails to satisfy the care coordination threshold, the subject is removed from the IBD care coordination program, when the resilience score fails to satisfy the care coordination threshold and the subject exhibits indications of low resilience, the subject is reassessed to determine an updated resilience score after a second predetermined period has elapsed, and when the resilience score continues to satisfy the care coordination threshold, the IBD care coordination program is continued.

In some embodiments, the predetermined period is at least a month, at least two months, at least three months, at least four months, at least six months, or between four months and eight months.

In some embodiments, the inflammatory bowel disease is ulcerative colitis or Crohn's disease.

In some embodiments, the inflammatory bowel disease is Crohn's disease and the IBD severity score is determined based on one or more criteria selected from the group consisting of: the American Gastroenterological Associations' (AGA) published risk stratification tool; current or history of perianal disease; presence or absence of moderate to severe rectal disease; presence or absence of extensive disease including ileal involvement greater than 40 cm or pancolitis; presence or absence of large or deep mucosal lesions; history of IBD related hospitalization within last 12 months; presence of stoma; prior intestinal resections; presence or absence of stricturing disease; presence of bowel fistulas (internal penetrating); steroid dependent disease or use within last 12 months; current or historical use of biologics, small molecules or immunomodulators for IBD management; and lack of symptomatic improvement with prior exposure to biologics and/or immunomodulators.

In some embodiments, the inflammatory bowel disease is Crohn's disease; the IBD severity score is determined based on one or more criteria selected from the group consisting of: the AGA risk stratification tool; current or history of perianal disease; presence or absence of moderate to severe rectal disease; presence or absence of extensive disease including ileal involvement greater than 40 cm or pancolitis; presence or absence of large or deep mucosal lesions; history of IBD related hospitalization within last 12 months; presence of stoma; prior intestinal resections; presence or absence of stricturing disease; presence of bowel fistulas (internal penetrating); steroid dependent disease or use within last 12 months; current or historical use of biologics, small molecules or immunomodulators for IBD management; and lack of symptomatic improvement with prior exposure to biologics and/or immunomodulators; and the IBD severity score is high-risk.

In some embodiments, the inflammatory bowel disease is ulcerative colitis and the IBD severity score is determined using one or more criteria selected from the group consisting of: the AGA risk stratification tool; deep ulcers on the subject's latest colonoscopy; history of IBD related hospitalization within last 12 months; steroid dependent disease or use within last 12 months; current or historical use of biologics, small molecules or immunomodulators for IBD management; and lack of symptomatic improvement with prior exposure to biologics, small molecules and/or immunomodulators.

In some embodiments, the inflammatory bowel disease is ulcerative colitis; the IBD severity score is determined using one or more criteria selected from the group consisting of: the AGA risk stratification tool; deep ulcers on the subject's latest colonoscopy; history of IBD related hospitalization within last 12 months; steroid dependent disease or use within last 12 months; current or historical use of biologics, small molecules or immunomodulators for IBD management; and lack of symptomatic improvement with prior exposure to biologics, small molecules and/or immunomodulators; and the IBD severity score is high-risk.

In some embodiments, the at least one time-limited care plan includes managing IBD medication for the subject, managing IBD related appointment keeping for the subject, tracking one or more IBD related health issues for the subject, or managing daily activities for the subject.

In some embodiments, the inflammatory bowel disease is ulcerative colitis (e.g., E1, proctitis; E2, left-sided; or E3, extensive).

In some embodiments, the inflammatory bowel disease is Crohn's disease (e.g., A1, disease onset ≤16 years; A2, disease onset 17-40 years; L1, terminal ileum; L2, colon; L3, ileocolon; L4, upper gastrointestinal; L1, L2, L3+L4; P, perianal disease modifier; B1, nonstructuring, nonpenetrating; B2, stricturing/B3, penetrating, etc.).

Another aspect of the present disclosure provides a non-transitory computer-readable storage medium for evaluating a subject for inflammatory bowel disease (IBD) care coordination strategy. The non-transitory computer readable storage medium stores instructions configured for execution by a computer system. When executed by the computer system, the instructions cause the computer system to determine a resilience score, within a range of resilience scores, for the subject, where each of a plurality of different resilience domain assessments of the subject contributes to the resilience score. The instructions further cause the computer system to use the resilience score to determine whether to enlist the subject in an IBD care coordination program. When the resilience score fails to satisfy a care coordination threshold, the subject is not enlisted in the IBD care coordination program. When the resilience score satisfies the care coordination threshold, the instructions further comprises performing the IBD care coordination program by a procedure comprising: determining an IBD severity score, within a range of IBD severity scores, for the subject based, at least in part, on a risk for one or more IBD complications or surgery to alleviate IBD, using both the resilience score and the IBD severity score to assign the subject into a category in a plurality of categories, where each category in the plurality of categories is associated with a unique combination of a sub-range of the range of resilience scores and a sub-range of the range of IBD severity scores, determining at least one time-limited care plan, at a first time point, for the subject based upon the identity of the assigned category within the plurality of categories, and prioritizing the at least one time-limited care plan by an outcome of one or more resilience domain assessments in the plurality of different resilience domain assessments.

Another aspect of the present disclosure provides a computer system for evaluating a subject for inflammatory bowel disease (IBD) care coordination strategy. The computer system comprises one or more processors, memory, and one or more programs stored in the memory for execution by the one or more processors. The one or more programs comprise instructions for determining a resilience score, within a range of resilience scores, for the subject, where each of a plurality of different resilience domain assessments of the subject contributes to the resilience score. The one or more programs further comprise instructions for using the resilience score to determine whether to enlist the subject in an IBD care coordination program. When the resilience score fails to satisfy a care coordination threshold, the subject is not enlisted in the IBD care coordination program. When the resilience score satisfies the care coordination threshold, the method further comprises performing the IBD care coordination program by a procedure comprising determining an IBD severity score, within a range of IBD severity scores, for the subject based, at least in part, on a risk for one or more IBD complications or surgery to alleviate IBD, using both the resilience score and the IBD severity score to assign the subject to a category in a plurality of categories, where each category in the plurality of categories is associated with a unique combination of a sub-range of the range of resilience scores and a sub-range of the range of IBD severity scores, determining at least one time-limited care plan, at a first time point, for the subject based upon the identity of the assigned category within the plurality of categories, and prioritizing the time-limited care plan by an outcome of one or more resilience domain assessments in the plurality of different resilience domain assessments.

Other embodiments are directed to systems, portable consumer devices, and computer readable media associated with the methods described herein. As disclosed herein, any embodiment disclosed herein can be applied in some embodiments to any other aspect. Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 illustrates a server storing instructions for methods described herein, in accordance with an embodiment of the present disclosure.

FIGS. 2A, 2B, and 2C collectively illustrate a flowchart outlining methods for evaluating a subject for inflammatory bowel disease management, in accordance with an embodiment of the present disclosure.

FIG. 3 illustrates factors involved in determining GRITT scores, in accordance with an embodiment of the present disclosure.

FIG. 4 illustrates example categories (e.g., quadrants 402e, 402f, 402g, 402h) into which patients can be categorized, in accordance with some embodiments of the present disclosure. Each category corresponds to a set of resilience scores (e.g., each category has a corresponding range of resilience scores) and a prediction of disease prognosis (e.g., a disease severity). The categories indicate which patients are at highest risk for complications and adverse events, and depending upon how patients are categorized, individualized care plans can be determined.

FIG. 5 illustrates the results of training patients in resilience, in accordance with an embodiment of the present disclosure. For example, the 126 patients who were deemed to be GRITT eligible (e.g., eligible to receive resilience training) and who completed the training, experienced 90% fewer emergency department (ED) visits and 88% fewer hospitalizations in the year after training than in the 6 months before resilience training. In contrast, the control group of 210 patients who were deemed GRITT eligible but who did not engage in the training experienced substantially the same number of ED visits and 65% more hospitalizations in the year after declining training than in the 6 months before being deemed eligible for training.

FIG. 6 illustrates example questions for determining a resilience score for a subject in accordance with an embodiment of the present disclosure.

FIG. 7 illustrates an example management pipeline, in accordance with an embodiment of the present disclosure.

FIG. 8 illustrates multiple factors involved in providing health care to patients in accordance with an embodiment of the present disclosure.

FIG. 9 illustrates multiple factors involved in evaluating IBD severity score for patients, in accordance with an embodiment of the present disclosure.

FIG. 10 illustrates an example workflow for determining whether to enlist a subject in an IBD care coordination program and implementation of an IBD care coordination program, in accordance with an embodiment of the present disclosure.

FIGS. 11A and 11B illustrate the results of enlisting subjects in an IBD care coordination program, in accordance with an embodiment of the present disclosure. For example, in FIG. 11A, the 184 patients who participated in the program had a 71% reduction in ED visits and a 94% reduction in hospitalizations after completing the training. Opioid use and steroid use were also significantly reduced (49% and 73% decrease, respectively). In contrast, as illustrated in FIG. 11B, the 210 patients that were considered historical controls did not differ from the patients who participated in the program with respect to demographics, clinical characteristics or mental health diagnoses, but exhibited only a 4% reduction in ED visits and a 16% reduction in hospitalizations.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for evaluating a subject for inflammatory bowel disease (IBD) management.

The terms “subject” and “patient” are used interchangeably herein and refer to a human who is known to have, or potentially has, a medical condition or disorder, such as, e.g., IBD. In some embodiments, a subject is a male or female of any stage (e.g., a man, a woman or a child).

Several aspects are described below with reference to example applications for illustration. It should be understood that numerous specific details, relationships, and methods are set forth to provide a full understanding of the features described herein. One having ordinary skill in the relevant art, however, will readily recognizes that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Exemplary System Embodiments

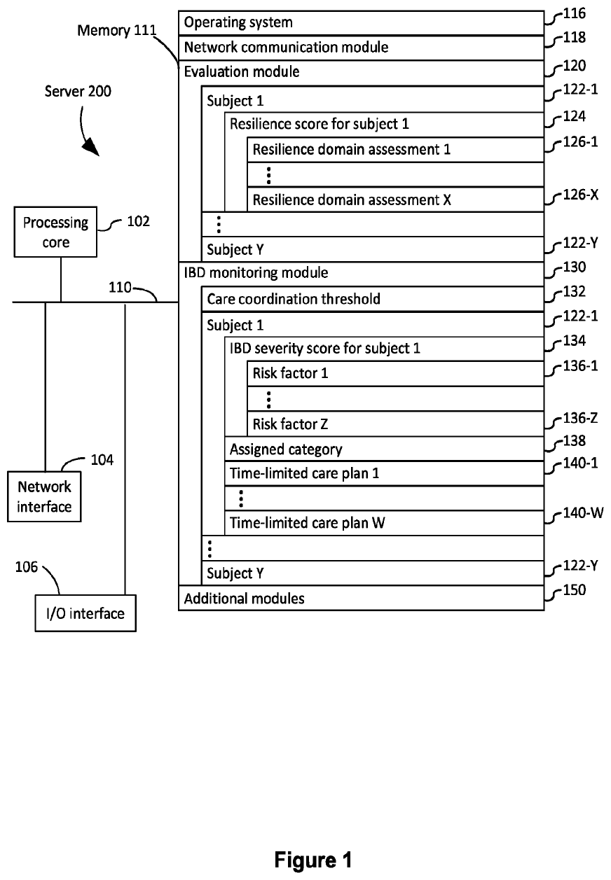

A detailed description of a system that can be used by medical practitioners to determine appropriate management plan courses for patients in accordance with the present disclosure is described in conjunction with FIG. 1. As such, FIG. 1 illustrates the topology of the system, including server 100 in accordance with the present disclosure. In some embodiments, server 100 includes one or more processing units CPU(s) 102 (also referred to as processors), one or more network interfaces 104, memory 111 for storing programs and instructions for execution by the one or more processors 102, one or more communications interfaces such as input/output interface 106, and one or more communications buses 110 for interconnecting these components.

The one or more communication buses 110 optionally include circuitry (sometimes called a chipset) that interconnects and controls communications between system components. The memory 111 typically includes high-speed random access memory, such as DRAM, SRAM, DDR RAM, ROM, EEPROM, flash memory, CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Memory 111 optionally includes one or more storage devices remotely located from the CPU(s) 102. Memory 111, and the non-volatile memory device(s) within the memory 111, comprise a non-transitory computer readable storage medium.

In some embodiments, memory 111 or alternatively the non-transitory computer readable storage medium stores the following programs, modules and data structures, or a subset thereof:
operating system 116, which includes procedures for handling various basic system services and for performing hardware dependent tasks;
network communication module (or instructions) 118 for connecting server 100 with other devices, or a communication network;
evaluation module 120, which determines, for a respective subject 122, a resilience score 124 that is based at least in part on one or more resilience domain assessments 126 for the subject;
an IBD monitoring module 130 for a) determining, from a respective subject's 122 resilience score 124, an IBD severity score 136 that is based at least in part on one or more risk factors 136 for the respective subject, b) assigning the respective subject 122 to an assigned category 138 based at least in part on the care coordination threshold 132, and c) determining at least one time-limited care plan 140 for the respective subject based on the subject's assigned category 138; and
additional modules 150 for performing additional evaluations, for providing information to health care providers, and/or for connecting subjects to appropriate care options.

In various implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, memory 111 optionally stores a subset of the modules and data structures identified above. Furthermore, in some embodiments, the memory stores additional modules and data structures not described above. In some embodiments, one or more of the above identified elements is stored in a computer system, other than that of server 100, that is addressable by server 100 so that server 100 may retrieve all or a portion of such data when needed.

Although FIG. 1 depicts server 100, the figure is intended more as a functional description of the various features that may be present in computer systems than as a structural schematic of the implementations described herein. In typical embodiments, a server 100 comprises one or more computers. However, the disclosure is not so limited. The functionality of server 100 may be spread across any number of networked computers and/or reside on each of several networked computers and/or by hosted on one or more virtual machines at a remote location accessible across the communications network 106. In practice, and as recognized by those of ordinary skill in the art, items shown separately could be combined and some items could be separated. One of skill in the art will appreciate that a wide array of different computer topologies is possible for server 100 and all such topologies are within the scope of the present disclosure.

### Specific Embodiments of the Disclosure

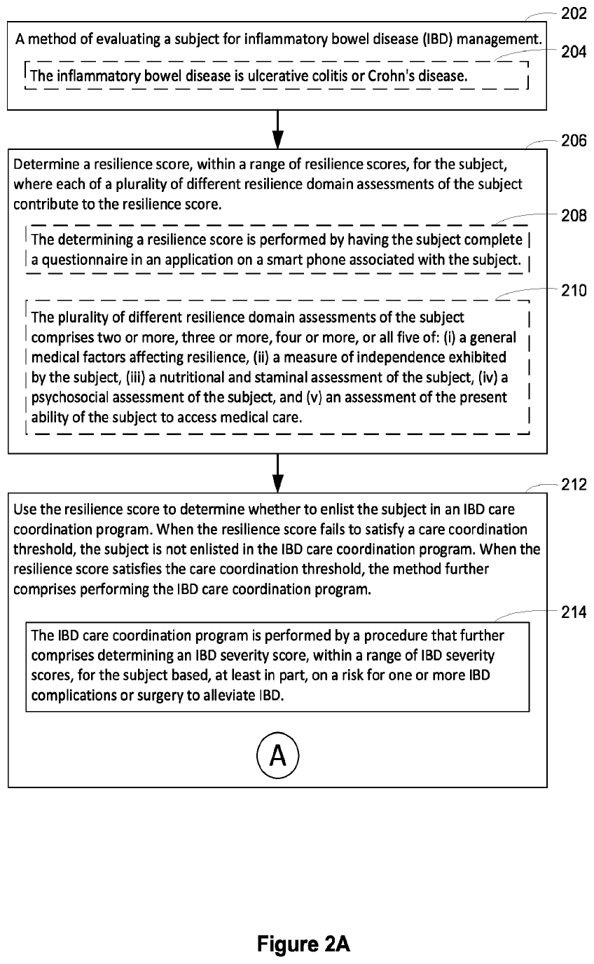

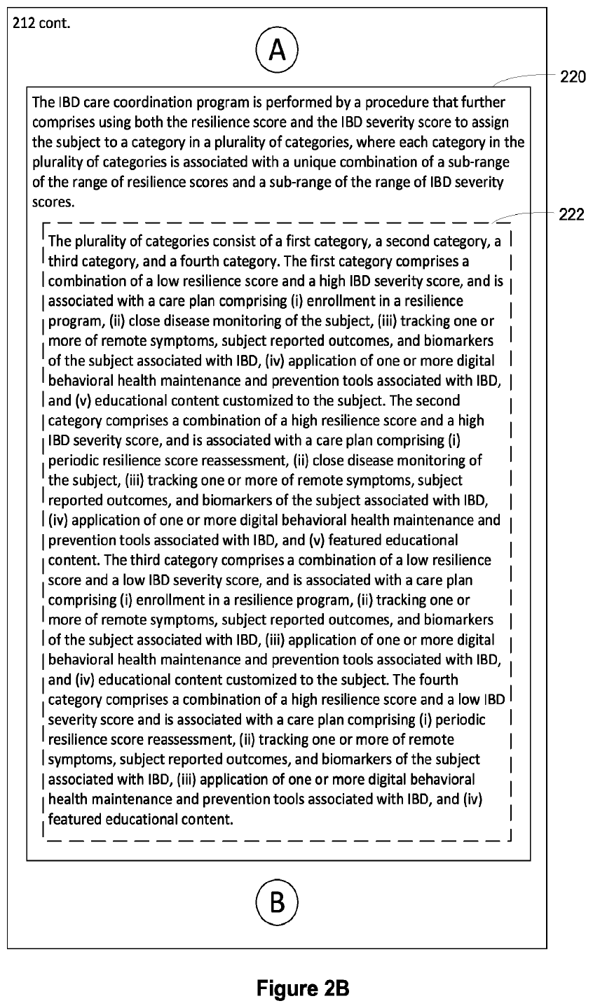

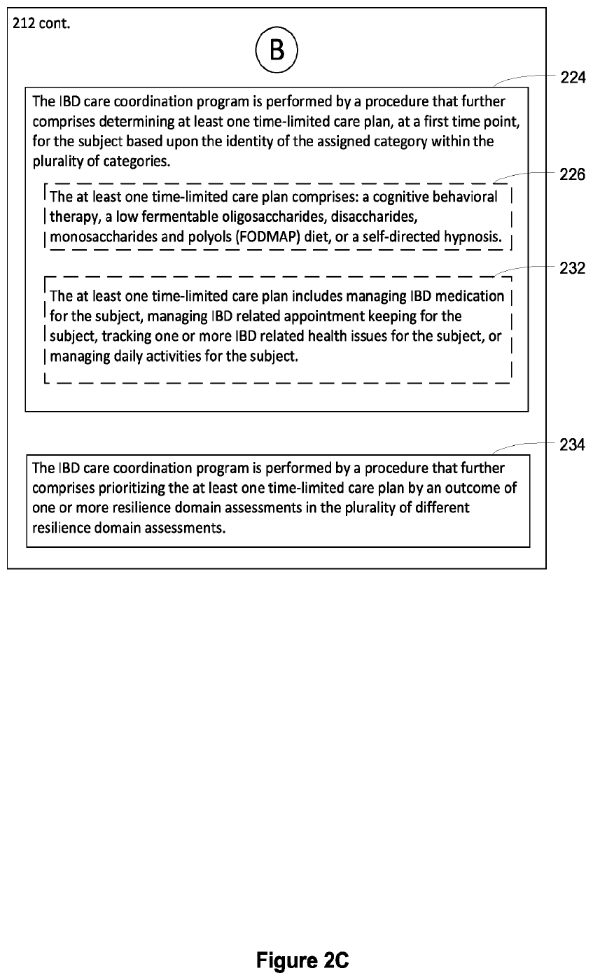

Now that details of server 100 have been disclosed, details regarding a flow chart of processes and features of the system, in accordance with an embodiment of the present disclosure, are disclosed with reference to FIGS. 2A-2C.

Block 202. Referring to block 202 in FIG. 2A, one aspect of the present disclosure provides a method that operates at a computer system, such as server 100, and has one or more processors and memory storing one or more programs to be executed by the one or more processors to perform the method. In the method, a subject is evaluated for inflammatory bowel disease management. In some embodiments, referring to block 204, the inflammatory bowel disease is ulcerative colitis or Crohn's disease.

Block 206. Referring to block 206, the method proceeds by determining a resilience score (e.g., a GRITT score), within a range of resilience scores, for the subject, where each of a plurality of different resilience domain assessments of the subject contribute to the resilience score. Resilience is defined herein as one's ability to physiologically and psychologically recover from adversity, maintain optimism, and regain well-being. In some embodiments, the range of resilience scores is from 0 to 100. In some embodiments, the range of resilience scores is from 0 to 10, from 1 to 10, from 1 to 100, or from 0 to 1. For example, GRITT scores are described in further detail herein (see, Example 3: Resilience training pipeline).

As used herein, the term “assessment” is used to refer to any examination and/or questioning of a subject diagnosed with IBD to monitor the progress of the IBD over time, study the effectiveness of therapies, or determine the IBD prognosis of the subject.

Referring to block 208, in some embodiments, the determination of a resilience score 124 (e.g., a GRITT score) is performed by having the subject complete a questionnaire in an application on a smart phone associated with the subject (e.g., the questionnaire is part of a mobile phone application). In some embodiments, the questionnaire is available for the subject in any input/output device 106 capable of communicating with server 100. In some embodiments, the questionnaire includes questions that are found in the Connor-Davidson Resilience Scale (CD-RISC), a validated 10-item questionnaire measuring one's perceived ability to bounce back from adversity. See, Campbell-Sills and Stein (2007), “Psychometric analysis and refinement of the Connor-Davidson Resilience Scale (CD-RISC): Validation of a 10-item Measure of Resilience,” J Trauma Stress. 2007; 20:1019-1028. In some embodiments, the questionnaire includes questions that are found in a Transition Readiness Assessment Questionnaire (TRAQ). See, Wood et al. (2014) “The transition Readiness Assessment Questionnaire (TRAQ): its factor structure, reliability, and validity,” Acad Peadiatr. 14, 415-422, which is hereby incorporated by reference. In some embodiments, one or more questions in the questionnaire are on a Likert scale. A Likert scale is a multipoint (e.g., five or seven, etc.) scale that is used to allow an individual to express how much they agree or disagree with a particular statement.

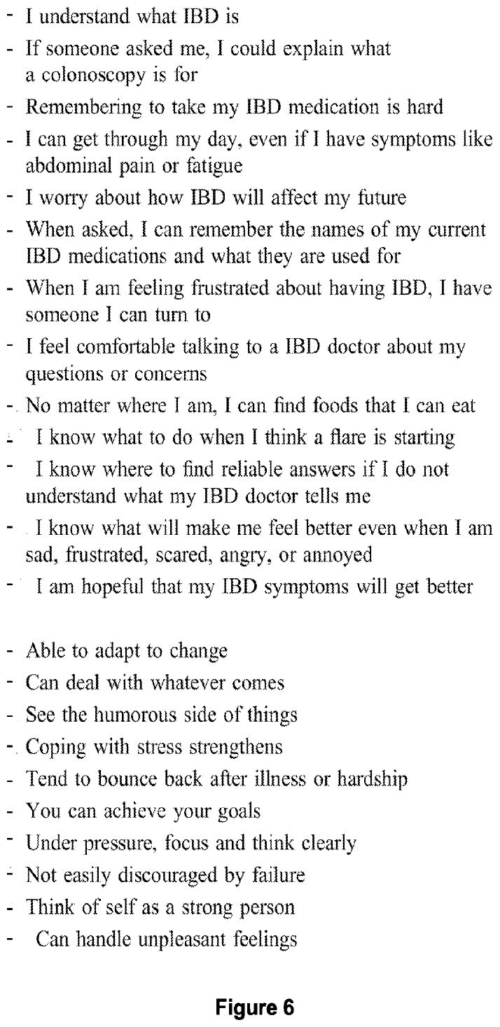

In some embodiments, the questionnaire comprises one or more, two or more, three or more, four or more, five or more, six or more, seven or more, eight or more, nine or more, ten or more, eleven or more, twelve or more, thirteen or more, fourteen or more, fifteen or more, twenty of the questions illustrated in FIG. 6. In some embodiments, the questionnaire comprises at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 60, at least 70, at least 80, at least 90, or at least 100 questions. In some embodiments, the questionnaire comprises no more than 200, no more than 100, no more than 80, no more than 70, no more than 60, no more than 50, no more than 40, no more than 30, or no more than 20 questions. In some embodiments, the questionnaire comprises from 5 to 25 questions, from 10 to 35 questions, from 8 to 60 questions, or from 20 to 100 questions. In some embodiments, the questionnaire comprises a plurality of questions that falls within another range starting no lower than 2 and ending no higher than 200 questions.

In some embodiments, the determination of a resilience score (e.g., a GRITT score) is performed by having the subject complete a questionnaire (e.g., in an application on a smart phone), where one or more questions in the questionnaire are used to assess one or more resilience domains for the subject. For instance, in some embodiments, for each respective resilience domain in a plurality of resilience domains, one or more questions in the questionnaire are directed to the respective resilience domain, where the response provided by the subject to the one or more questions generates an assessment of the respective resilience domain for the subject. In some such embodiments, the resilience score (e.g., the GRITT score) considers the assessments (e.g., outcomes) of all of the resilience domains obtained from the responses of the subject.

In some embodiments, each of the plurality of different resilience domain assessments of the subject contribute to the resilience score equally. In some embodiments, one or more resilience domain assessments in the plurality of different resilience domain assessments of the subject contribute to the resilience score unequally (e.g., where one or more resilience domain assessments are weighted for the determination of the resilience score).

Referring to block 210, in some embodiments, the plurality of different resilience domain assessments 126 of the subject comprises at least one, two or more, three or more, four or more, or all five of: (i) general medical factors affecting resilience, (ii) a measure of independence exhibited by the subject, (iii) a nutritional and staminal assessment of the subject, (iv) a psychosocial assessment of the subject, and (v) an assessment of the present ability of the subject to access medical care. Each of these domains interrogates the overall medical status of the subject.

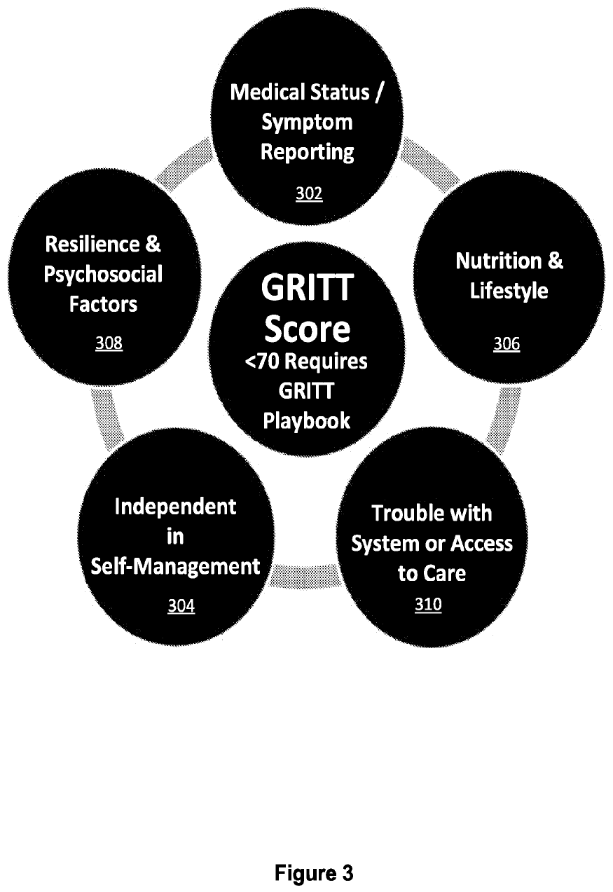

FIG. 3 illustrates potential resilience domains used for assessing the subject's GRITT score. For example, in some embodiments, general medical factors affecting resilience (e.g., 302 “medical status/symptom reporting”) comprise one or more of currently active IBD, high symptom reporting, ongoing comorbidities, and/or complications, IBD surgery within the previous year, and health care utilization (e.g., number of hospitalization and/or emergency department (ED) visits within the past 6 months). In some embodiments, general medical factors affecting resilience further include current disease activity (e.g., as determined by the Harvey Bradshaw Index (HBI) and/or Mayo Score). In some embodiments, general medical factors affecting resilience further include diagnoses (e.g., IBD type and/or symptoms).

In some embodiments, the measure of independence exhibited by the subject (e.g., 304 “independence in self-management”) comprises lack of disease acceptance, relationship difficulties with caregivers or care team, difficulty attending work/school, and/or whether the subject either no shows or frequently cancels medical appointments.

In some embodiments, the nutritional and staminal assessment of the subject (e.g., 306 “nutrition & lifestyle”) comprises significant weight change/loss, fat or muscle depletion, liquid nutrient diet or intravenous nutrition, and/or amount of physical activity/exercise.

In some embodiments, the psychosocial assessment of the subject (e.g., 308 “resilience & psychosocial factors”) comprises subject having trouble handling stress, depression/anxiety symptoms in subject, symptoms of chronic pain, prior mental health issues, substance abuse, and/or any psychotropic use. For instance, in some embodiments, psychosocial assessments further include data related to opioid use (e.g., yes/no) and/or steroid use (e.g., yes/no) during a period of time prior to data collection and/or enrollment in a care plan.

In some embodiments, the assessment of the present ability of the subject to access medical care (e.g., 310 “trouble with system or access to care”) comprises the subject currently having a primary care provider, subject satisfaction with care, subject requiring additional care coordination, subject lacking access to reliable transportation, residential instability of the subject, and/or subject having financial/insurance concerns. Each of these resilience domain assessments serve to evaluate the ability of the subject to manage their IBD effectively. In some embodiments, a subject may score highly in one or more of the resilience domain assessments, in two or more of the resilience domain assessments, in three or more of the resilience domain assessments, in four or more of the resilience domain assessments, or in all five of the abovementioned resilience domain assessments. Additional resilience domain assessments are possible, as described further below and as will be apparent to one skilled in the art. In some embodiments, a patient scores highly in at least 1, at least 2, at least 3, at least 4, at least 5, at least 6, at least 7, at least 8, at least 9, at least 10, at least 15, or at least 20 resilience domain assessments.

In some embodiments, the plurality of different resilience domain assessments 126 of the subject comprises the subject's experience of physical health, and are one or more of: (i) when the subject is in remission, a report of high impact of symptoms on daily functions, (ii) when the subject has had at least one unplanned hospitalization or emergency department (ED) visit in the previous 12 months, (iii) when the subject has had an IBD-related surgery in the previous 6 months, (iv) when the subject will have surgery in the next 6 months, and (v) a report of chronic pain by the subject. In some embodiments, the IBD-related surgery is a proctocolectomy (removal of entire colon and rectum), an ileoanal anastomosis (removal of colon and creation of a pouch inside the body that connects the small intestine to the anal canal). In some embodiments, the IBD-related surgery is ileo-cecal resection and primary reconstruction, stricturoplasty, and endoscopic dilatations of jejunum and ileum. See, for example, Sica and Biancone (2013), “Surgery for inflammatory bowel disease in the era of laparoscopy,” World J. Gastroenterol 19(16), 2445-2448, which is hereby incorporated by reference.

In some embodiments, the plurality of different resilience domain assessments 126 of the subject comprises the subject's experience of physical health in the form of chronic pain, fatigue, urgency to use the bathroom, incontinence, nausea, and recent or future health care use. In some embodiments, the plurality of different resilience domain assessments of the subject further comprises the subject's experience of other health conditions. In some embodiments, any health conditions known to co-occur with IBD are included in the plurality of different resilience domain assessments.

In some embodiments, the plurality of different resilience domains further includes demographics data (e.g., age, sex, race, ethnic background, and/or insurance type). In some embodiments, the plurality of different resilience domains further includes additional clinical history and/or drug use (e.g., biologic use). In some instances, any of the different assessments disclosed herein are not exclusively associated with any one respective resilience domain, but can be obtained in combination with any other assessment or in the context of any other resilience domain as will apparent to one skilled in the art.

For example, where the plurality of different resilience domain assessments is obtained using a questionnaire, in some embodiments, different questions in the questionnaire are used to assess different factors (e.g., various general medical factors affecting resilience).

In some embodiments, different resilience domain assessments are collected at any time prior, during, or post-enrollment in a care program or regimen. For instance, in some embodiments, data collection for resilience domain assessments is performed at least 2 years, at least 1 year, at least 8 months, at least 6 months, at least 4 months, at least 3 months, at least 2 months, at least 1 month, or at least 1 week prior to enrollment. In some embodiments, data collection for resilience domain assessments is performed at start of enrollment and/or at completion of a care program. In some embodiments, data collection for resilience domain assessments is performed at intervals during participation of a care program (e.g., at least every 6 months, at least every 4 months, at least every 3 months, at least every 2 months, at least every month, at least every 2 weeks, at least every week, or at least every day). In some embodiments, data collection for resilience domain assessments is performed at least 1 week, at least 1 month, at least 2 months, at least 3 months, at least 4 months, at least 6 months, at least 8 months, at least 1 year, or at least 2 years after completion of a care program.

In some embodiments, a resilience domain assessment is obtained at multiple time points (e.g., at regular intervals and/or at fixed, predetermined time points). For instance, in some embodiments, one or more resilience domain assessments are obtained for a subject on at least 2, at least 3, at least 4, at least 5, at least 6, at least 7, at least 8, at least 9, at least 10, at least 15, at least 20, at least 25, at least 30, at least 35, at least 40, at least 45, or at least 50 separate occasions.

In some embodiments, assessments are used to collect data for various resilience domains occurring at any point in a patient's timeline (e.g., at any point in a patient's clinical history) prior to a time point of interest (e.g., the date of assessment, a date of enrollment in a care program, and/or a date of completion of a care program). For example, resilience domain assessments can be used to query a patient's clinical history up to 3 months prior, up to 6 months prior, up to 1 year prior, up to 2 years prior, up to 5 years prior, or more than 5 years prior to the time point of interest.

For example, in an embodiment, a resilience domain assessment is used to determine whether a patient used opioids or steroids within a period of 1 year prior to data collection. In such embodiment, this assessment is performed at time of enrollment in a care program and 12 months after completion of the care program.

In some embodiments, a plurality of resilience scores is determined for a respective subject at a plurality of time points. For example, in some embodiments, an updated resilience score is determined each time a resilience domain assessment is obtained. Thus, in some instances where one or more resilience domain assessments are obtained at a plurality of time points, the resilience score for the subject is also determined at each of the plurality of time points.

Block 212. Referring to block 212, the method continues by using the resilience score 124 to determine whether to enlist the subject in an IBD care coordination program. When the resilience score 124 fails to satisfy a care coordination threshold (e.g., when the subject's resilience score is higher than the care coordination threshold), the subject is not enlisted in the IBD care coordination program. When the resilience score 124 satisfies the care coordination threshold (e.g., when the subject's resilience score falls below the care coordination threshold), the method further comprises performing the IBD care coordination program.

In some embodiments, the care coordination threshold comprises a score of at least 50, at least 55, at least 60, at least 65, at least 70, at least 75, at least 80, at least 85, at least 90, or at least 95 (e.g., where if the subject's score is below 50, below 55, below 60, below 65, below 70, below 75, below 80, below 85, below 90, or below 95, respectively, the subject is enlisted in the IBD care coordination program). In some embodiments the care coordination threshold is determining the resilience score for each subject in a cohort of IBD subjects and choosing a care coordination threshold based on the mean resilience score of the cohort, or a standard deviation thereof. For instance, in some embodiments, the care coordination threshold is one standard deviation below the mean, the mean, or one standard deviation above the mean.

In some embodiments, the care coordination threshold is based on an outcome for a resilience domain assessment disclosed herein. For example, in some embodiments, the care coordination threshold is based on a risk of unplanned care for patients with IBD (e.g., ED visits and/or hospitalizations).

Referring to block 214, the IBD care coordination program is performed by a procedure that further comprises determining an IBD severity score 134, within a range of IBD severity scores, for the subject based, at least in part, on a risk for one or more IBD complications or surgery to alleviate IBD (e.g., risk factors 136). For instance, in some embodiments, an IBD severity score indicates the risk to the subject of having complications due to IBD and/or of requiring surgery to treat IBD (e.g., in the absence of an IBD care coordination program).

Block 220. Referring to block 220 of FIG. 2B, the IBD care coordination program is performed by a procedure that further comprises using both the resilience score 124 and the IBD severity score 134 to assign the subject to a category 138 in a plurality of categories. Each category in the plurality of categories is associated with a unique combination of a sub-range of the range of resilience scores and a sub-range of the range of IBD severity scores (e.g., a first category in the plurality of categories is associated with a first sub-range of the range of resilience scores and a first sub-range of the range of IBD severity scores, a second category in the plurality of categories is associated with a second sub-range of the range of resilience scores and a second sub-range of the range of IBD severity scores, etc.).

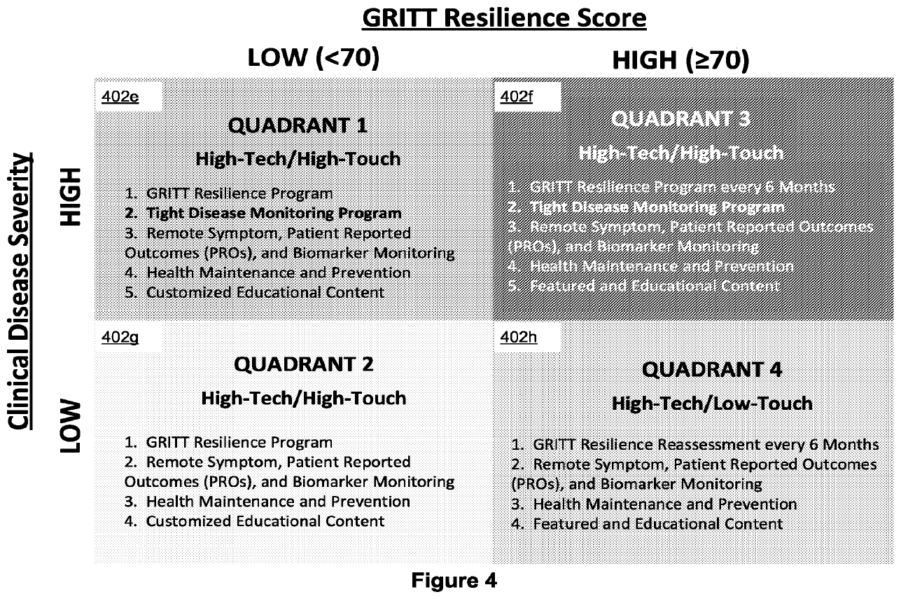

Referring to block 222, in some embodiments, the plurality of categories consists of a first category (e.g., quadrant 402e in FIG. 4), a second category (e.g., quadrant 402f), a third category (e.g., quadrant 402g), and a fourth category (e.g., quadrant 402h). In some embodiments, the first category comprises a combination of a low resilience score and a high IBD severity score, and is associated with a care plan comprising (i) enrollment in a resilience program, (ii) close disease monitoring of the subject, (iii) tracking one or more of remote symptoms, subject reported outcomes, and biomarkers of the subject associated with IBD, (iv) application of one or more digital behavioral health maintenance and prevention tools associated with IBD, and (v) educational content customized to the subject. In some embodiments, the second category comprises a combination of a high resilience score and a high IBD severity score, and is associated with a care plan comprising (i) periodic resilience score reassessment, (ii) close disease monitoring of the subject, (iii) tracking one or more of remote symptoms, subject reported outcomes, and biomarkers of the subject associated with IBD, (iv) application of one or more digital behavioral health maintenance and prevention tools associated with IBD, and (v) featured educational content. In some embodiments, the third category comprises a combination of a low resilience score and a low IBD severity score, and is associated with a care plan comprising (i) enrollment in a resilience program, (ii) tracking one or more of remote symptoms, subject reported outcomes, and biomarkers of the subject associated with IBD, (iii) application of one or more digital behavioral health maintenance and prevention tools associated with IBD, and (iv) educational content customized to the subject. In some embodiments, the fourth category comprises a combination of a high resilience score and a low IBD severity score and is associated with a care plan comprising (i) periodic resilience score reassessment, (ii) tracking one or more of remote symptoms, subject reported outcomes, and biomarkers of the subject associated with IBD, (iii) application of one or more digital behavioral health maintenance and prevention tools associated with IBD, and (iv) featured educational content. For example, in some embodiments, the subjects in category 4 are at lowest risk and thus require only low levels of technological and medical monitoring.

In some instances, the different categories require different levels of monitoring. For example, in some embodiments, a subject is categorized into a quadrant based on a determination of a behavioral health prognosis (e.g., low resilience or high resilience) and a determination of a disease prognosis (e.g., a disease severity, such as a low risk or a moderate-to-high risk for IBD complications and surgery). In some instances, a subject that is determined to have a low resilience score and either a low risk or a moderate-to-high risk for disease severity is associated with a care plan comprising both high technological monitoring and high medical monitoring (e.g., high technical and high touch). In some embodiments, a subject that is determined to have a high resilience score and a moderate-to-high risk for disease severity is associated with a care plan comprising medium levels of both technological and medical monitoring (e.g., medium tech and medium touch). In some instances, a subject that is determined to have a high resilience score and a low risk for disease severity is associated with a care plan comprising low levels of both technological and medical monitoring (e.g., low tech and low touch).

In some embodiments, the care plan prescribed to a subject based on an assigned category (e.g., risk stratification using resilience score and disease severity) comprises any combination of therapies, programs, monitoring and/or educational services suitable for the respective category, as will be apparent to one skilled in the art. For instance, as illustrated in FIG. 4, in some embodiments, a subject that is determined to have a low resilience score (e.g., less than 70) and a high risk for clinical disease severity (e.g., quadrant 1 (402e)) is associated with a care plan comprising both high technological monitoring and high medical monitoring (e.g., high tech and high touch). In some instances, the care plan for the subject with low resilience and high disease severity comprises (1) enrollment in a GRITT resilience program, (2) a tight disease monitoring program, (3) monitoring of remote symptoms, patient reported outcomes (PRO), and biomarkers, (4) health maintenance and prevention, and (5) customized educational content.

In some embodiments, a subject that is determined to have a low resilience score (e.g., less than 70) and a low risk for clinical disease severity (e.g., quadrant 2 (402g)) is associated with a care plan comprising both high technological monitoring and high medical monitoring (e.g., high tech and high touch). In some instances, the care plan for the subject with low resilience and low disease severity comprises (1) enrollment in a GRITT resilience program, (2) monitoring of remote symptoms, patient reported outcomes (PRO), and biomarkers, (3) health maintenance and prevention, and (4) customized educational content.

In some embodiments, a subject that is determined to have a high resilience score (e.g., greater than or equal to 70) and a high risk for clinical disease severity (e.g., quadrant 3 (402f)) is associated with a care plan comprising both high technological monitoring and high medical monitoring (e.g., high tech and high touch). In some instances, the care plan for the subject with low resilience and high disease severity comprises (1) GRITT resilience reassessment every 6 months, (2) a tight disease monitoring program, (3) monitoring of remote symptoms, patient reported outcomes (PRO), and biomarkers, (4) health maintenance and prevention, and (5) featured and educational content.

In some embodiments, a subject that is determined to have a high resilience score (e.g., greater than or equal to 70) and a low risk for clinical disease severity (e.g., quadrant 4 (402h)) is associated with a care plan comprising both high technological monitoring and low medical monitoring (e.g., high tech and low touch). In some instances, the care plan for the subject with low resilience and low disease severity comprises (1) GRITT resilience reassessment every 6 months, (2) monitoring of remote symptoms, patient reported outcomes (PRO), and biomarkers, (3) health maintenance and prevention, and (4) featured and educational content.

In some embodiments, technological monitoring (e.g., high tech) comprises the use of mobile applications for delivery of services. In some embodiments, medical monitoring (e.g., high touch) comprises the use of a licensed care teach for delivery of services. For example, in some embodiments, medical monitoring comprises the assistance of a personal GRITT coach and/or the integration of additional health care professionals into the subject's coordinated care plan, such as a multidisciplinary care team and/or a health psychologist. In some embodiments, health care professionals participate in selection of care plans, monitoring of patient progress, and assessment of satisfactory or unsatisfactory treatment effects.

In some embodiments, the plurality of categories comprises any possible category based on any combination of stratified resilience scores and/or stratified IBD severity scores. In some embodiments, stratified scores are characterized by an indication of degree (e.g., low, medium-low, medium-high, high) or a plurality of cutoff thresholds (e.g., 0-25, 25-50, 50-75, 75-100, etc.).

Thus, in some embodiments, the plurality of categories comprises at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 categories.

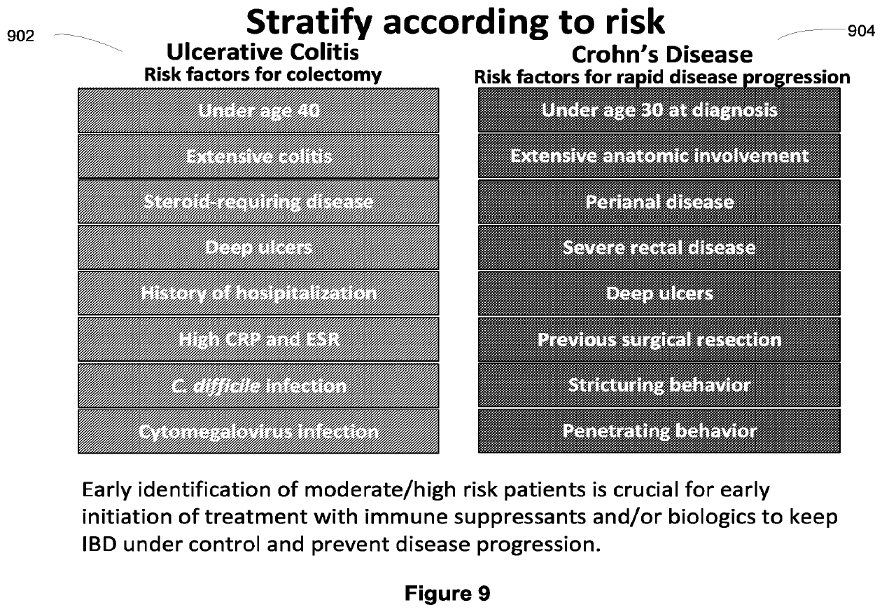

In some embodiments, the IBD risk of the subject (e.g., the IBD severity score of the subject) is determined (e.g., as part of determining an appropriate coordinated care plan for the subject) for either Crohn's disease or ulcerative colitis. FIG. 9 illustrates some of the risk factors involved in categorizing (e.g., stratifying) the subject with regards to ulcerative colitis 902 or Crohn's disease 904 (e.g., there are different risk factors for each diagnosis). The various risk factors included in FIG. 9 are from the published American Gastroenterological Association risk stratification tool. In some embodiments, the IBD severity score for the subject is determined using the American Gastroenterological Associations' (AGA) published risk stratification tool. See Sandborn (2014) “Crohn's disease evaluation and treatment: clinical decision tool” Gastroenterol 147, 702-705 for a description of the AGA stratification tool specific to Crohn's disease and the components therein. See Dassopoulos et al. (2015) “Ulcerative Colitis Care Pathway” Gastroenterol 149, 238-245 for a description of the AGA stratification tool that is specific to ulcerative colitis.

In some embodiments, the IBD severity score for the subject is determined using alternative risk stratification tools (e.g., including different sets of risk factors and/or weighing subject responses differently). In some embodiments, the IBD severity score for the subject is determined using any risk stratification tool.

For example, in some embodiments, the inflammatory bowel disease is Crohn's disease and the IBD severity score is determined based on one or more criteria selected from the group consisting of: the AGA risk stratification tool; current or history of perianal disease; presence or absence of moderate to severe rectal disease; presence or absence of extensive disease including ileal involvement greater than 40 cm or pancolitis; presence or absence of large or deep mucosal lesions; history of IBD related hospitalization within last 12 months; presence of stoma; prior intestinal resections; presence or absence of stricturing disease; presence of bowel fistulas (internal penetrating); steroid dependent disease or use within last 12 months; current or historical use of biologics, small molecules or immunomodulators for IBD management; and lack of symptomatic improvement with prior exposure to biologics and/or immunomodulators.

In some embodiments, the one or more criteria disclosed above is determined by a clinician or a medical practitioner. In some embodiments, the presence of any one of the above criteria will result in the determination of a high severity score (e.g., a “high risk” score).

For instance, in some embodiments, the inflammatory bowel disease is Crohn's disease; the IBD severity score is determined based on one or more criteria selected from the group consisting of: the AGA risk stratification tool; current or history of perianal disease; presence or absence of moderate to severe rectal disease; presence or absence of extensive disease including ileal involvement greater than 40 cm or pancolitis; presence or absence of large or deep mucosal lesions; history of IBD related hospitalization within last 12 months; presence of stoma; prior intestinal resections; presence or absence of stricturing disease; presence of bowel fistulas (internal penetrating); steroid dependent disease or use within last 12 months; current or historical use of biologics, small molecules or immunomodulators for IBD management; and lack of symptomatic improvement with prior exposure to biologics and/or immunomodulators; and the IBD severity score is high-risk.

In some embodiments, the inflammatory bowel disease is ulcerative colitis and the IBD severity score is determined using one or more criteria selected from the group consisting of: the AGA risk stratification tool; deep ulcers on the subject's latest colonoscopy; history of IBD related hospitalization within last 12 months; steroid dependent disease or use within last 12 months; current or historical use of biologics, small molecules or immunomodulators for IBD management; and lack of symptomatic improvement with prior exposure to biologics, small molecules and/or immunomodulators.

In some embodiments, the one or more criteria disclosed above is determined by a clinician or a medical practitioner. In some embodiments, the presence of any one of the above criteria will result in the determination of a high severity score (e.g., a “high risk” score).

For instance, in some embodiments, the inflammatory bowel disease is ulcerative colitis; the IBD severity score is determined using one or more criteria selected from the group consisting of: the AGA risk stratification tool; deep ulcers on the subject's latest colonoscopy; history of IBD related hospitalization within last 12 months; steroid dependent disease or use within last 12 months; current or historical use of biologics, small molecules or immunomodulators for IBD management; and lack of symptomatic improvement with prior exposure to biologics, small molecules and/or immunomodulators; and the IBD severity score is high-risk.

It is possible to determine a coordinated care plan for a subject at any stage of IBD. In some embodiments, the inflammatory bowel disease is Crohn's disease (e.g., A1, disease onset ≤16 years; A2, disease onset 17-40 years; L1, terminal ileum; L2, colon; L3, ileocolon; L4, upper gastrointestinal; L1, L2, L3+L4; P, perianal disease modifier; B1, nonstricturing, nonpenetrating; B2, stricturing/B3, penetrating, etc.). In some embodiments, the inflammatory bowel disease is Crohn's disease (e.g., A1, disease onset ≤16 years; A2, disease onset 17-40 years; L1, terminal ileum; L2, colon; L3, ileocolon; L4, upper gastrointestinal; L1, L2, L3+L4; P, perianal disease modifier; B1, nonstricturing, nonpenetrating; B2, stricturing/B3, penetrating, etc.). In some embodiments, the inflammatory bowel disease is ulcerative colitis (e.g., E1, proctitis; E2, left-sided; or E3, extensive).

In some embodiments, one or more biomarkers are used, at least in part, for determining the IBD risk of the subject (e.g., for subjects at different stages of IBD). In particular, biomarkers can be used to determine diagnosis and/or disease prognosis. See e.g., Zeng et al. (2019) “From Genetics to Epigenetics, Roles of Epigenetics in Inflammatory Bowel Disease” Front Genet doi: 10.3389/fgene.2019.01017; Norouzinia et al. (2017) “Biomarkers in inflammatory bowel diseases: insight into diagnosis, prognosis and treatment” Gastroenterol Hepatol Bed Bench 10(3), 155-167; and Zhou et al. (2018) “Gut Microbiota Offers Universal Biomarkers across Ethnicity in Inflammatory Bowel Disease Diagnosis and Infliximab Response Prediction” mSystems 3:e00188-17.

In some embodiments, the inflammatory bowel disease is ulcerative colitis, and the one or more biomarkers comprises one or more, two or more, or three or more of the biomarkers in the group consisting of: C-reactive protein (CRP), fecal calprotectin, hemoglobin, and albumin.

In some embodiments, the inflammatory bowel disease is Crohn's disease, and the one or more biomarkers comprises one or more, two or more, or three or more of the biomarkers in the group consisting of: C-reactive protein (CRP), fecal calprotectin, hemoglobin, and albumin.

In some embodiments, the one or more biomarkers further comprises fecal lactoferrin and/or fecal neopterin.

Other methods and tools for determining severity scores (e.g., diagnosis, prognosis, and/or risk) are possible, as will be apparent to one skilled in the art. Various tools and indices for determining severity scores include, but are not limited to, the Harvey Bradshaw Index (HBI), Mayo Score/Disease Activity Index (DAI), Crohn's Disease Activity Index (CDAI), van Hees Index, Perianal Disease Activity Index (PDAI), Inflammatory Bowel Disease Questionnaire (IBDQ), Manitoba IBD Index, numeric rating scale, IBD-Control questionnaire, CD Digestive Damage Score/Lemann index, UC Disease Activity Index (UCDAI), Rachmilewitz Score/Clinical Activity Index (CAI), Powell-Tuck Index/St. Mark's Index, abbreviated Powell-Tuck Index, Simple Clinical Colitis Activity Score/Walmsley Score (SCGAI), Lichtiger Index, Seo Index, Endoscopic-Clinical Correlation Index, Crohn's Disease Endoscopic Index of Severity (CDEIS), Simple Endoscopic Score for Crohn's Disease (SES-CD), Ulcerative Colitis Endoscopic Index of Severity (UCEIS), Lewis Score, Sonographic Lesion Index for CD, Tsuga colorectal ultrasound criteria, Riley scale, Geboes score, modified Riley and Geboes scale, Histologic Activity Index, Endocytoscopy System Score, Beaugerie Index/St. Antoine Model, and/or international definitions of the European Crohn's and Colitis Organisation (ECCO), the American College of Gastroenterology (ACG), and/or the Japanese Society of Gastroenterology (JSG). Methods and tools for determining severity scores (e.g., diagnosis, prognosis, and/or risk) are further described, for example, in Biroulet et al., 2016, “Defining Disease Severity in Inflammatory Bowel Diseases: Current and Future Directions,” Clin Gastro Hep 14(3): 348-354.e17; doi: 10.1016/j.cgh.2015.06.001, which is hereby incorporated herein by reference in its entirety.

Referring to block 224, the IBD care coordination program procedure further comprises determining at least one time-limited care plan, at a first time point, for the subject based upon the identity of the assigned category within the plurality of categories. In some embodiments, the IBD care coordination program procedure further comprises determining a plurality of time-limited care plans, at the first time point, for the subject based on the identity of the assigned category within the plurality of categories.

In some embodiments, the determining the at least one time-limited care plan comprises determining, based upon the identity of the assigned category, one or more desired resilience goals in a plurality of resilience goals for the subject.

In some embodiments, the plurality of resilience goals is selected from the group consisting of disease acceptance; realistic optimism; social support; self-confidence/self-efficacy; and self-regulatory skills.

In some embodiments, a resilience goal in the plurality of resilience goals is selected based upon an outcome of one or more resilience domain assessments obtained for the subject. In some embodiments, a resilience goal in the plurality of resilience goals is selected based on its projected ability to target (e.g., improve an outcome for) any of the different resilience domains used to obtain resilience domain assessments. For instance, in some embodiments, a resilience goal comprises an intervention or a service that is directed towards an improvement in general medical factors of the subject, independence exhibited by the subject, nutrition and stamina, psychosocial characteristics of the subject (e.g., social work), and/or ability of the subject to access medical care.

In some embodiments, one or more resilience goals in the plurality of resilience goals are transdiagnostic. Thus, in some instances, any of the plurality of resilience goals disclosed herein are not exclusively associated with any one specialization but aim to address or improve a plurality of resilience domains or a subcategory of a resilience domain. For instance, a care plan with nutrition targeting self-efficacy can focus on healthier nutrition choices, social work targeting self-efficacy can focus on bathroom anxiety, and pharmacy targeting self-efficacy can focus on self-injection. As described below, in some embodiments, targets are chosen based on strengths and limitations identified in the resilience score and/or in the resilience domain assessments.

For example, in some embodiments, the one or more resilience goals for a respective subject comprises a change (e.g., a % increase) in resilience score (e.g., GRITT score) for the subject over the course of enrollment in a care plan.

In some embodiments, the plurality of different resilience domain assessments of the subject comprises a measure of health care utilization (e.g., number of ED visits and/or hospitalizations). In some such embodiments, the one or more resilience goals comprises a change in (e.g., a % reduction) in ED visits and hospital admissions for the subject.

In some embodiments, the plurality of different resilience domain assessments of the subject comprises a measure of independence exhibited by the subject that indicates that the subject has inadequate independence. In some embodiments, this measure of independence is determined in the resilience questionnaire through questions posed to the subject regarding degree of lack of disease acceptance, relationship difficulties with caregivers or care team, difficulty in attending work or school, frequency of skipping scheduled medical appointments on a Likert scale.

In some embodiments, applying the at least one time-limited care plan to the subject comprises arranging to have the subject meet with a social worker and/or a clinical pharmacist who is associated with the time-limited care plan. In some embodiments, the subject meets with one or more health care professionals (e.g., a social worker, a clinical pharmacist, a multidisciplinary care team and/or a health psychologist) during a plurality of sessions. In some embodiments, the plurality of sessions comprises at least 3, at least 4, at least 5, at least 6, at least 7, at least 8, at least 9, at least 10, at least 11, at least 12, at least 13, at least 14, at least 15, at least 20, at least 25, at least 30, at least 40, or at least 50 sessions. In some embodiments, the plurality of sessions comprises no more than 100, no more than 50, no more than 40, no more than 30, or no more than 20 sessions. In some embodiments, the plurality of sessions comprises from 2 to 12, from 5 to 20, from 2 to 30, or from 5 to 80 sessions. In some embodiments, the plurality of sessions falls within a different range starting no lower than 2 sessions and ending no higher than 100 sessions.

In some embodiments, the at least one time-limited care plan comprises therapy or one or more lifestyle changes. Referring to block 226, in some embodiments, the at least one time-limited care plan comprises a cognitive behavioral therapy (See, e.g., Mikocka-Walus, 2017, “Cognitive-Behavioural Therapy for Inflammatory Bowel Disease: 24-Month Data from a Randomised Controlled Trial,” Int. J. Behav. Med. 24, 127-135), a low fermentable oligosaccharides, disaccharides, monosaccharides and polyols (FODMAP) diet (See, e.g., Gibson and Shepherd, 2005, “Personal view: food for thought—western lifestyle and susceptibility to Crohn's disease, The FODMAP hypothesis,” Alimentary Pharmacology & Therapeutics. 21, 1399-40), or a self-directed hypnosis. Examples of self-directed hypnosis include, but are not limited to, hypnotherapy and, in particular, gut-directed hypnotherapy. Gut-directed hypnotherapy is a form of medical hypnosis that draws upon metaphors and delivers post-hypnotic suggestions specific to the improved health and function of the gastrointestinal tract. See, e.g., Keefer et al. (2013), “Gut-directed hypnotherapy significantly augments clinical remission in quiescent ulcerative colitis,” Aliment Pharmacol Ther. 38(7), pp. 761-771, which is hereby incorporated by reference.

Referring to block 232, in some embodiments, at least one time-limited care plan includes managing IBD medication for the subject (e.g., providing dosage recommendations, providing medication recommendations, tracking potential drug-drug interactions, etc.), managing IBD related appointment keeping for the subject (e.g., automatic appointment scheduling, providing appointment reminders, etc.), tracking one or more IBD related health issues for the subject (e.g., keeping records on secondary conditions affected by the subject's IBD), or managing daily activities for the subject (e.g., activity scheduling, sending daily activity reminders to the subject, etc.).

In some embodiments, an intervention or service directed towards achieving a resilience goal is performed in person (e.g., as in-patient or outpatient care). In some embodiments, an intervention or service directed towards achieving a resilience goal is performed via telehealth.

In some embodiments, the IBD care coordination program procedure further comprises determining a plurality of time-limited care plans, where each of the care plans in the plurality of care plans is selected using one or more desired resilience goals determined based upon the identity of the assigned category and/or upon the outcome of resilience domain assessments obtained for the subject. In some implementations, each time-limited care plan in the plurality of time-limited care plans comprises any of the embodiments of care plans disclosed herein, or any modifications, substitutions, additions, deletions, or combinations thereof, as will be apparent to one skilled in the art.

In some embodiments, the plurality of time-limited care plans comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, or at least 50 care plans. In some embodiments, the plurality of time-limited care plans comprises no more than 100, no more than 50, no more than 40, no more than 30, no more than 20, or no more than 10 care plans. In some embodiments, the plurality of time-limited care plans comprises from 3 to 20, from 5 to 15, from 2 to 10, or from 10 to 100 care plans. In some embodiments, the plurality of time-limited care plans comprises a different range starting no lower than 2 care plans and ending no higher than 100 care plans.

Block 234. Referring to block 234, in some embodiments, the IBD care coordination program procedure further comprises prioritizing the at least one time-limited care plan by an outcome of one or more resilience domain assessments in the plurality of different resilience domain assessments. In some such embodiments, the outcome of one or more resilience domain assessments indicates which time-limited care plans would be most beneficial to the subject's health.

In some embodiments, for example when there are a plurality of time-limited care plans (e.g., at least 2, at least 3, at least 4, at least 5, at least 6, at least 7, at least 8, at least 9, or at least 10 time-limited care plans), the prioritizing ensures that the plurality of care plans do not interfere with each other. For example, in some embodiments, two or more time-limited care plans in the plurality of time-limited care plans have contradictory treatments. In some embodiments, two or more time-limited care plans in the plurality of time-limited care plans have parallel treatments (e.g., administration of similar medications).

For example, in some embodiments, the plurality of different resilience domain assessments of the subject comprises a measure of independence exhibited by the subject that indicates that the subject has inadequate independence (e.g., for patients who may have difficulties with remembering their medication regimens or for patients who are unable to access health care providers). In some such embodiments, the method further comprises applying the at least one time-limited care plan to the subject by arranging to have the subject meet with a social worker and/or a clinical pharmacist associated with the assigned care plan.

In this way, the method provides a personalized, integrated care plan that is selected based on specific targets for a respective subject, where the specific targets are indicated by the subject's responses to one or more resilience domain assessments. Thus, targets for treatment by the at least one time-limited care plan are chosen based on the strengths and limitations of the respective subject identified in the determining of the resilience score (e.g., the baseline GRITT score).

Example processes for identifying a subject for IBD management and for enrolling the patient in a coordinated care plan, in accordance with some embodiments of the present disclosure, are provided in further detail below in Example 3.

IBD is a chronic disease, and thus many patients will require long-term monitoring in order to manage their disease successfully. Thus, in some embodiments, the method further comprises repeating the determining the resilience score after a first predetermined period has elapsed since the first time point (e.g., enrollment in a care plan). In some such embodiments, when the resilience score fails to satisfy the care coordination threshold, the subject is removed from the IBD care coordination program, when the resilience score fails to satisfy the care coordination threshold and the subject exhibits indications of low resilience, the subject is reassessed to determine an updated resilience score after a second predetermined period has elapsed, and when the resilience score continues to satisfy the care coordination threshold, the IBD care coordination program is continued. In some embodiments, the method further comprises repeating the obtaining a plurality of different resilience domain assessments after a first predetermined period has elapsed since the first time point (e.g., enrollment in the care plan). In some such embodiments, when the outcome of each respective resilience domain assessment in the plurality of resilience domain assessments satisfies a corresponding optimal outcome threshold, the subject is removed from the IBD care coordination program (e.g., all targets are optimized by the care plan), and when an outcome of a resilience domain assessment fails to satisfy an optimal outcome threshold for the respective resilience domain assessment, the IBD care coordination program is continued.

In some embodiments, the first and/or the second predetermined period is at least a day, at least two days, at least a week, at least two weeks, at least a month, at least two months, at least three months, at least four months, at least six months, between a day and two weeks, between a week and two months, between one month and four months, between four months and eight months, or between six months and a year. In some embodiments, the predetermined period is at least six months, at least seven months, at least eight months, at least nine months, at least ten months, at least eleven months, or at least a year. For example, in some embodiments, if a subject has a resilience score that fails to satisfy the care coordination threshold but has an indication of low resilience (e.g., due to a relatively low resilience score that nevertheless does not satisfy the care coordination threshold, and/or one or more outcomes from a corresponding one or more resilience domain assessments), then the subject is reexamined to determine a resilience score (e.g., a GRITT score) after a further period of time has elapsed (e.g., 6 months).

In some embodiments, the subject's continued enrollment in a care plan and the duration thereof is determined, monitored, and/or updated by one or more health care professionals (e.g., a multidisciplinary care team (MCT) and/or a health psychologist). In some embodiments, the monitoring is performed at a plurality of time points (e.g., intervals). In some embodiments, the determining, monitoring and/or updating is performed at least daily, at least weekly, at least bi-weekly, or at least monthly. In some embodiments, the determining, monitoring and/or updating is performed after one or more predetermined periods disclosed above.

In some embodiments, the subject is enrolled in a care plan for at least 1 week, at least 2 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 18 months, or at least 2 years prior to completion of the program. In some embodiments, the subject is enrolled in a care plan for no more than 5 years, no more than 4 years, no more than 3 years, no more than 2 years, no more than 1 year, no more than 8 months, no more than 6 months, or no more than 3 months prior to completion of the program. In some embodiments, the subject is enrolled in a care plan for from 1 month to 2 years, from 3 months to 18 months, from 6 months to 14 months, from 4 months to 1 year, or from 1 year to 3 years prior to completion of the program. In some embodiments, the subject is enrolled in a care plan for another period of time starting no lower than 1 week and ending no higher than 5 years.

By using a combination of the subject's resilience (e.g., through the resilience score 124) and the subject's likelihood of developing complications (e.g., IBD severity score), the methods described herein provide improved care plans (e.g., treatment suggestions) and thus improved outcomes to the subject (see, e.g., Example 1).

### Additional Specific Embodiments of the Disclosure

The following clauses describe specific embodiments of the disclosure.

Clause 1. A method of evaluating a subject for management of an inflammatory bowel disease (IBD), the method comprising: determining a resilience score, within a range of resilience scores, for the subject, wherein each of a plurality of different resilience domain assessments of the subject contribute to the resilience score; and using the resilience score to determine whether to enlist the subject in an IBD care coordination program, wherein: when the resilience score fails to satisfy a care coordination threshold, the subject is not enlisted in the IBD care coordination program, and when the resilience score satisfies the care coordination threshold, the method further comprises performing the IBD care coordination program by a procedure comprising: determining an IBD severity score, within a range of IBD severity scores, for the subject based, at least in part, on a risk for one or more IBD complications or surgery to alleviate IBD; using both the resilience score and the IBD severity score to assign the subject to a category in a plurality of categories, wherein each category in the plurality of categories is associated with a unique combination of a sub-range of the range of resilience scores and a sub-range of the range of IBD severity scores; determining at least one time-limited care plan, at a first time point, for the subject based upon the identity of the assigned category within the plurality of categories; and prioritizing the at least one time-limited care plan by an outcome of one or more resilience domain assessments in the plurality of different resilience domain assessments.

Clause 2. The method of clause 1, where: the plurality of categories consists of a first category, a second category, a third category, and a fourth category, wherein: the first category comprises a combination of a low resilience score and a high IBD severity score, and is associated with a care plan comprising (i) tracking one or more biomarkers of the subject associated with IBD, (ii) application of one or more therapeutics associated with IBD, (iii) close digital monitoring of the subject, and (iv) assigning a personal coach and care team to the subject to monitor care plan progress; the second category comprises a combination of a high resilience score and a high IBD severity score, and is associated with a care plan comprising tracking one or more biomarkers of the subject associated with IBD; the third category comprises a combination of a low resilience score and a low IBD severity score, and is associated with a care plan comprising (i) application of one or more therapeutics associated with IBD (ii) and close digital monitoring of the subject; and the fourth category comprises a combination of a high resilience score and a low IBD severity score and is associated with a care plan that provides low levels of technological and medical monitoring of the subject associated with IBD.

Clause 3. The method of clause 1, wherein the plurality of different resilience domain assessments of the subject comprises two or more, three or more, four or more, or all five of: (i) a general medical factor of the subject affecting resilience, (ii) a measure of independence exhibited by the subject, (iii) a nutritional and staminal assessment of the subject, (iv) a psychosocial assessment of the subject, and (v) an assessment of the present ability of the subject to access medical care.

Clause 4. The method of clause 3, wherein the plurality of different resilience domain assessments of the subject comprises a general medical factor of the subject, is one or more of: (i) when the subject is in remission, a report of high impact of one or more physical symptoms on a plurality of daily functions, (ii) when the subject has had at least one unplanned hospitalization or emergency department visit in the previous twelve months, (iii) when the subject has had an IBD-related surgery in the previous six months, (iv) when the subject has surgery planned within the next six months, and (v) a report of chronic pain by the subject.

Clause 5. The method of clause 1, wherein the plurality of different resilience domain assessments of the subject comprises the subject's experience of physical health in the form of chronic pain, fatigue, urgency to use the bathroom, incontinence, nausea and recent or future health care use.

Clause 6. The method of clause 3, wherein the plurality of different resilience domain assessments of the subject comprises a measure of independence exhibited by the subject, and the subject is deemed to have inadequate independence when the subject is afraid to self-inject or undergo infusion or a necessary medical procedure, has a poor relationship with a caregiver or care team, is a child or young adult, has a poor attendance record at work or school, or has frequently cancelled or failed to attend medical appointments.

Clause 7. The method of clause 1, wherein: the plurality of different resilience domain assessments of the subject comprises a measure of independence exhibited by the subject that indicates that the subject has inadequate independence; and the one time-limited care plan comprises arranging to have the subject meet with a social worker and/or a clinical pharmacist associated with the one time-limited care plan.

Clause 8. The method of clause 1, wherein the at least one time-limited care plan comprises: a cognitive behavioral therapy; a low fermentable oligosaccharides, disaccharides, monosaccharides and polyols (FODMAP) diet; or a self-directed hypnosis.

Clause 9. The method of clause 1, wherein the determining a resilience score is performed by having the subject complete a questionnaire in an application on a smart phone associated with the subject.

Clause 10. The method of clause 1, wherein the procedure further comprises repeating the determining the resilience score after a predetermined period has elapsed since the first time point, wherein: when the resilience score fails to satisfy the care coordination threshold, the subject is removed from the IBD care coordination program; and when the resilience score continues to satisfy the care coordination threshold, the IBD care coordination program is continued.

Clause 11. The method of clause 10, wherein the predetermined period is at least a month, at least two months, at least three months, at least four months, at least six months, or between four months and eight months.

Clause 12. The method of clause 1, wherein the inflammatory bowel disease is ulcerative colitis or Crohn's disease.

Clause 13. The method of clause 1, wherein the inflammatory bowel disease is Crohn's disease and the IBD severity score is determined using the AGA risk stratification tool.

Clause 14. The method of clause 2, wherein: the inflammatory bowel disease is Crohn's disease; the IBD severity score is determined using the AGA risk stratification tool; and the high IBD severity score is medium/high-risk.

Clause 15. The method of clause 1, wherein the inflammatory bowel disease is ulcerative colitis and the IBD severity score is determined using the AGA risk stratification tool.

Clause 16. The method of clause 2, wherein: the inflammatory bowel disease is ulcerative colitis; the IBD severity score is determined using the AGA risk stratification tool; and the high IBD severity score is high-risk.

Clause 17. The method of clause 1, wherein the at least one time-limited care plan includes managing IBD medication for the subject, managing IBD related appointment keeping for the subject, tracking one or more IBD related health issues for the subject, or managing daily activities for the subject.

Clause 18. A non-transitory computer readable storage medium for evaluating a subject for management of an inflammatory bowel disease (IBD), wherein the non-transitory computer readable storage medium stores instructions, which when executed by a computer system, cause the computer system to: determine a resilience score, within a range of resilience scores, for the subject, wherein each of a plurality of different resilience domain assessments of the subject contribute to the resilience score; and use the resilience score to determine whether to enlist the subject in an IBD care coordination program, wherein: when the resilience score fails to satisfy a care coordination threshold, the subject is not enlisted in the IBD care coordination program, when the resilience score satisfies the care coordination threshold, the instructions further comprise performing the IBD care coordination program by a procedure comprising: determining an IBD severity score, within a range of IBD severity scores, for the subject based, at least in part, on a risk for one or more IBD complications or surgery to alleviate IBD; using both the resilience score and the IBD severity score to assign the subject into a category in a plurality of categories, wherein each category in the plurality of categories is associated with a unique combination of a sub-range of the range of resilience scores and a sub-range of the range of IBD severity scores; determining at least one time-limited care plan, at a first time point, for the subject based upon the identity of the assigned category within the plurality of categories; and prioritizing the at least one time-limited care plan by an outcome of one or more resilience domain assessments in the plurality of different resilience domain assessments.

Clause 19. A computer system for evaluating a subject for management of an inflammatory bowel disease (IBD), comprising: one or more processors; memory; and one or more programs stored in the memory for execution by the one or more processors, the one or more programs comprising instructions for: determining a resilience score, within a range of resilience scores, for the subject, wherein each of a plurality of different resilience domain assessments of the subject contribute to the resilience score; using the resilience score to determine whether to enlist the subject in an IBD care coordination program, wherein: when the resilience score fails to satisfy a care coordination threshold, the subject is not enlisted in the IBD care coordination program, and when the resilience score satisfies the care coordination threshold, the method further comprises performing the IBD care coordination program by a procedure comprising: determining an IBD severity score, within a range of IBD severity scores, for the subject based, at least in part, on a risk for one or more IBD complications or surgery to alleviate IBD; using both the resilience score and the IBD severity score to assign the subject to a category in a plurality of categories, wherein each category in the plurality of categories is associated with a unique combination of a sub-range of the range of resilience scores and a sub-range of the range of IBD severity scores; determining at least one time-limited care plan, at a first time point, for the subject based upon the identity of the assigned category within the plurality of categories; and prioritizing the time-limited care plan by an outcome of one or more resilience domain assessments in the plurality of different resilience domain assessments.

### EXAMPLES

### Example 1: The Effect of Resilience on Adverse Events in IBD Patients

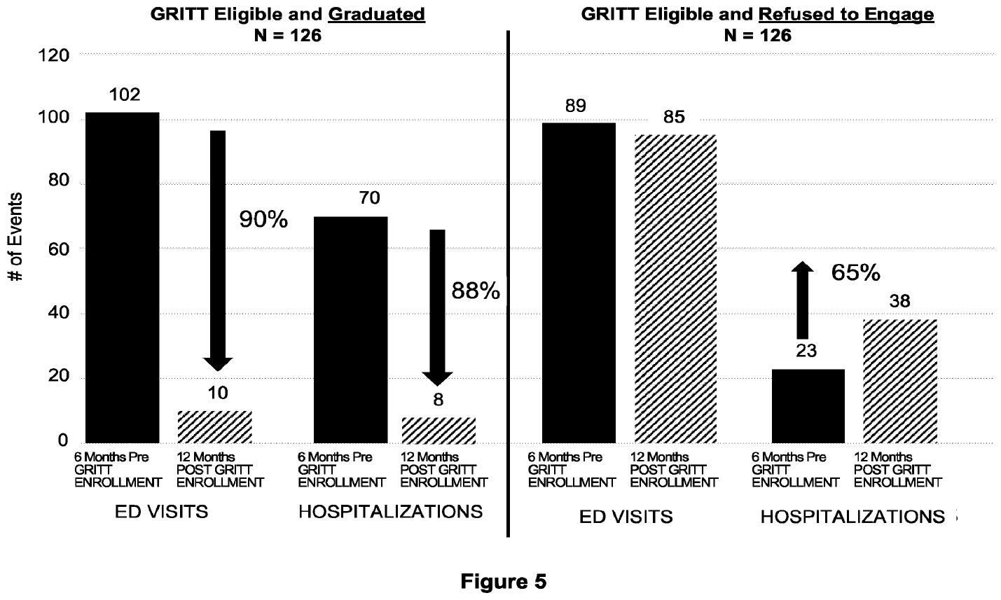

Patient resilience has a clear impact on the trajectory of IBD. Specifically, as shown in FIG. 5, patients who receive training to increase their resilience scores experience fewer adverse events. In FIG. 5, patients who were eligible were offered resilience training, and those who accepted were enrolled (e.g., were provided with training materials and/or received in-person instruction on techniques to increase resilience from health care providers). There were 126 patients who participated in resilience training (e.g., test group) and 210 patients who were eligible but who did not engage in training (e.g., control group). The test and control group were evaluated for ED visits and hospitalization pre-training (e.g., “pre-GRITT Enrollment” or “pre-GRITT Eligibility”) and post-training (e.g., “post-GRITT Enrollment” or “post-GRITT Refusal”). As shown in FIG. 5, for test group, ED visits decreased 90% and hospitalizations decreased 88% in the year after GRITT enrollment compared to the 6 months prior to GRITT enrollment. For comparison, in the control group, the number of ED visits remained similar while hospitalization increased 65% in the year after the patients declined GRITT enrollment compared to the 6 months prior to the patients' being deemed GRITT-eligible. Thus, there are distinct advantages for patients to improving resilience. By engaging in resilience training, patients can learn skills necessary to successfully manage chronic IBD.

### Example 2: Additional Effects of Resilience on Unplanned Care and Opioid Use in IBD Patients

### INTRODUCTION

Inflammatory bowel diseases, including Crohn's Disease and ulcerative colitis affect up to 3 million Americans with incidence in Western regions (US, UK, Canada) stabilizing around 1% but with incidence in young people and developing countries on the rise (1,2). Despite its relatively low prevalence, IBD is one of the costliest chronic conditions given the innate high costs of inpatient and outpatient management including biologic medication use, recurrent need for surgery and frequent hospitalizations (3). The costs of preventable, unplanned care in IBD is particularly concerning, with about 15% of IBD patients accounting for nearly 50% of the total costs of care (3, 4).

Unplanned IBD care has been directly linked to the presence of depression, anxiety and substance abuse (4), with a large claims database study demonstrating that patients with one or more mental health comorbidities cost the health system 3-5× more than their non-diagnosed counterparts (5). When mental health comorbidities are identified and addressed in an integrated behavioral health model, unplanned care is substantially reduced, supporting the hypothesis that unplanned care is a modifiable factor in cost containment and underscores the need for effective behavioral solutions in IBD management (6-8).

True rates of mental health comorbidity in IBD remain speculative due to limitations in study design but can be conservatively estimated at 30% for anxiety and 25% for depression (9). Most cases of anxiety and depression develop after the diagnosis of IBD (10), presumably due to the impact a serious diagnosis has on a person's daily life and function (11). A recent study of a large, longitudinal Swedish IBD cohort showed that depression was most likely to occur in the first year of diagnosis (12), supporting the hypothesis that a change in medical status and new self-management demands could increase vulnerability to the development of a mental health comorbidity, which, if not addressed could further drive higher rates of surgery, hospitalizations, disease flares, escalation in therapies and worsen mental health outcomes and quality of life (13).

In some embodiments, therefore, long-term disease outcomes in IBD can be impacted by considering the pre-clinical attitudes, beliefs and behaviors of patients based on their prior life experiences, goals, values and cultural background rather than waiting for them to screen positive on a depression or anxiety questionnaire (14). These pre-clinical strengths and limitations set the stage for disease self-management behaviors and can be an earlier target for behavioral intervention. For example, patients who are able to approach their condition and its changing disease demands over time with resilience, the ability to thrive despite adversity, are less likely to develop anxiety in the setting of their IBD (15) and feel less stigmatized by their condition (16). Further, lack of disease acceptance or poor self-regulatory skills (17, 18), high emotional representation of illness and low disease-related self-efficacy (19, 20) are associated with poor emotional and physical outcomes, including higher perceived disability, higher health care utilization and poorer IBD quality of life (21). Furthermore, low resilience has been linked to higher need for surgery in Crohn's Disease, poorer quality of life and higher disease activity (22), and is predictive of inflammation and disease flares in ulcerative colitis (23), with one study suggesting low resilience could even be a risk factor for a diagnosis of IBD (24).

Integrated behavioral health care remains a critical opportunity to contain costs of unplanned care in IBD but needs to go further in its support of patients in advance of a mental health diagnosis and failed disease self-management. Changing disease-interfering behaviors, attitudes and beliefs in real-time can foster healthy adjustment and resilience and allow for lifelong self-management behaviors that limit the impact of IBD on one's potential. The following paragraphs illustrate the effectiveness of integrated behavioral GI care programs on unplanned care and patient well-being (7, 25) and describe the implementation and impact of a resilience-based integrated care methodology that blends expertise in psychogastroenterology, the application of health psychology principles to digestive disorders, and positive psychology, the scientific study of why people flourish in the face of adversity (26). This scientific approach to IBD care is called the Gaining Resilience through Transitions (GRITT) Method, as described in greater detail below (see, Example 3: Resilience training pipeline).

Methods.

Consecutive patients seen in an academic IBD center with Crohn's Disease, ulcerative colitis or indeterminate colitis were screened for biopsychosocial complexity (low resilience) over a period of 43 months. Patients determined to have low resilience, as measured by the validated clinician-administered GRITT score (described below), were eligible for enrollment in the multidisciplinary team based program which included an integrated care plan (playbook) and follow-up sessions, provided over a 6-12 month period, through telehealth or in-person, with licensed experts in medical social work, clinical pharmacy, IBD nutrition, and health psychology. Eligibility also required that the patient had been seen in the IBD Center over the past 12 months in order to have available health utilization data and were planning to stay with the Center over the next year. Patients who required higher level psychiatric care (e.g., eating disorders, suicidality, personality disorders, substance abuse, serious mental illness) were referred for care outside of the program. Eligible patients were allocated into 2 groups: GRITT Graduates (enrolled in program for greater than 6 months) and GRITT Historical Controls (eligible but did not enroll, matched for age, gender and payer type).

Data was collected for each patient, including clinical information, demographics (e.g., age, sex, race or ethnic background, insurance type), IBD type, biologic use, current disease activity (e.g., Harvey Bradshaw Index (HBI), Mayo Score), and health care utilization (number of emergency department visits and/or number of hospitalizations, in the year prior and 12 months later). Data on opioid use (yes/no), and steroid use (yes/no) in the year prior to enrollment were collected at baseline and then again at 12 months post enrollment for the GRITT Graduate group only.

GRITT Scores were calculated at baseline for all participants and at “graduation” for the group of patients who participated in the program.

Analyses.

All demographic, clinical and resilience data were entered into SPSSv26 (Chicago, Ill.) for analyses. Descriptive statistics, including frequency, percentages and means/medians were calculated for all patients who were eligible for enrollment (regardless of engagement) and a one-way ANOVA with follow-up independent sample t-tests, along with chi-square for categorical variables were conducted to determine differences between graduates and historical controls on clinical and demographic variables as well as outcome. A repeated measures MANCOVA (Group X Time) was conducted to determine change in health care use (ED and hospitalizations) over time between GRITT Graduates and controls, controlling for baseline. The primary outcome was change in (% reduction) in ED and hospital admissions between groups at 1-year follow-up. A secondary outcome was change in (% reduction) in resilience (GRITT Score) over time among GRITT Graduates.

Results.

456 patients were screened for eligibility. 394 patients were eligible for enrollment based on resilience score <70. 184 participated in the program, including the 126 patients who participated in resilience training (e.g., test group) from Example 1 above, and the 210 patients who were eligible but who did not engage in training (e.g., control group) from Example 1 above were considered historical controls. The two groups did not differ with respect to demographics, clinical characteristics or mental health diagnoses. Primary reasons for non-engagement were geographic barriers (38%), limited insurance coverage/high deductible (30%), not interested (24%) and 8% were lost to follow-up. Baseline GRITT Scores were slightly statistically higher, but not clinically significantly different in the control group (54.2 vs 46.3, p=ns). Baseline ED visits and hospitalizations were numerically lower in the control group. Participant data is presented in Table 1.

TABLE 1
Comparison of Groups on Demographic Variables

| | | | |
|---|---|---|---|
| | GRITT | GRITT Historical | |
| | Graduates | Controls | |
| Characteristics | (N = 184) | (N = 210) | p-value |

Demographics

| | | | | | |
|---|---|---|---|---|---|
| Sex at birth is female | 106 | (58%) | 113 | (54%) | ns |
| Median Age (Range) | 35 | (18-71) | 36 | (18-74) | ns |
| % White, Non-Hispanic | 131 | (71%) | 143 | (68%) | ns |
| % Commercially insured | 149 | (81%) | 153 | (73%) | ns |

Disease Characteristics

| | | | | | |
|---|---|---|---|---|---|
| % Crohn's Disease | 119 | (65%) | 124 | (59%) | ns |
| % Biologic use | 130 | (71%) | 143 | (68%) | ns |
| % baseline any opioid use | 39 | (20%) | 42 | (20%) | ns |
| % baseline current prednisone use | 48 | (26%) | 63 | (30%) | ns |
| Median Baseline Harvey Bradshaw Index | 7 | (0-27) | 8 | (0-24) | ns |
| Median Baseline Mayo Score | 4 | (0-12) | 3 | (0-10) | ns |

Mental Health

| | | | | | |
|---|---|---|---|---|---|
| Baseline GRITT Score | 46.3 | (10.1) | 54.2 | (16.7) | p < .05* |
| % with a mental health diagnosis [F] code in | 59 | (32%) | 71 | (34%) | ns |
| EMR | | | | | |

GRITT Graduates spent a median number of 200 days (range 184-365 days) in the program and received a median of 8 (range 2-12) sessions with a multidisciplinary care team (MCT), the majority (80%) of which were completed in the first 6 months of enrollment. The most common services used were social work and nutrition. More than 45% of services were conducted via telehealth (all data was collected pre-COVID-19 pandemic).

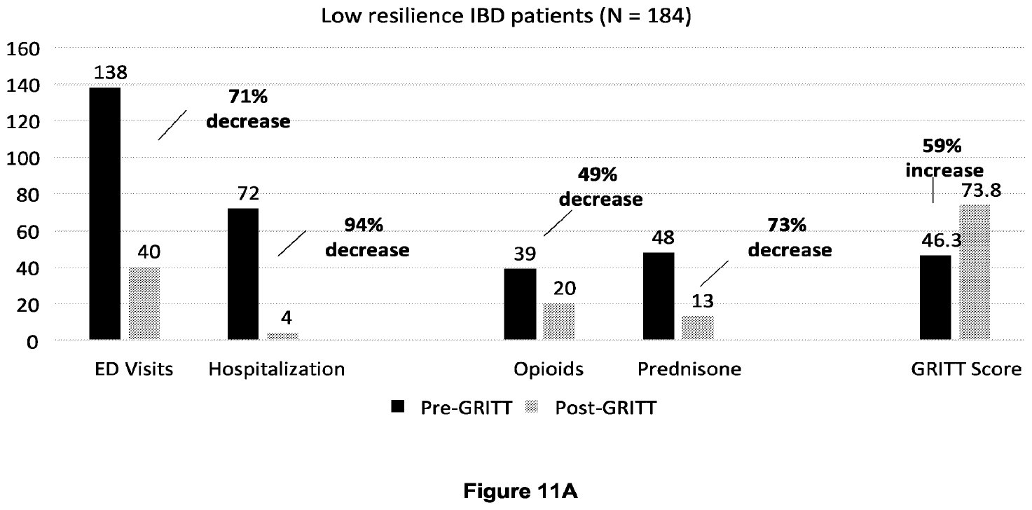

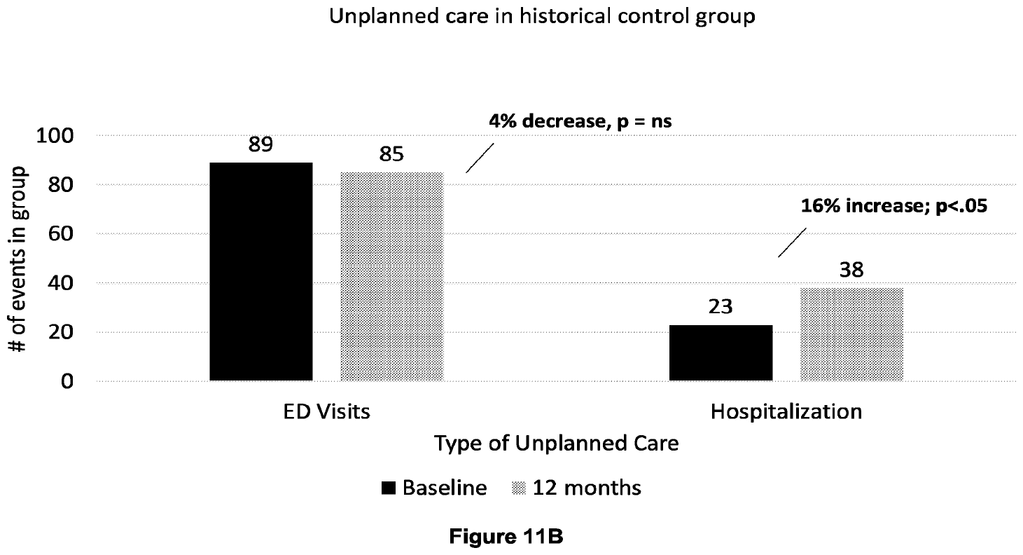

There was a significant group X time interaction such that the GRITT group had a 71% reduction in ED visits and a 94% reduction in hospitalizations compared to a 4% reduction in ED visits and a 16% reduction in hospitalizations among historical controls [F(1, 391)=421.6, p=0.000]. Opioid use and steroid use were also significantly reduced (49% and 73% decrease respectively) in the GRITT Group (data not available in controls) (p<0.001). Mean post-graduation GRITT score was 73.6 (8.3) an increase of 27.3 points (59% improvement) [p<0.000; 95% CI 29.3; 25.2], with a large effect size (cohen's d=2.4 [p<0.000; 95% CI 27, 2.0]. Health care outcomes of patients with and without participation in the care coordination (GRITT) program are illustrated in FIGS. 11A and 11B.

### DISCUSSION

Evaluation of a resilience-based method for reducing unplanned care among IBD patients with low resilience demonstrated substantial reduction in hospitalizations, emergency department visits, opioid and steroid use while simultaneously seeing a 2 standard deviation increase in resilience among patients engaged in the program. These results clearly demonstrate that a resilience-based approach can improve outcomes among patients with mental health comorbidities, and/or among patients who do not necessarily have a mental health comorbidity but who do have behavioral risk factors that interfere with optimal disease self-management. In fact, only about one third of our patients had a formal mental health comorbidity listed in their electronic medical record, suggesting that, in some embodiments, a resilience-based program is applicable to a wider range of IBD patients, and in some instances can be used to prevent a mental health diagnosis and its associated burden while still improving outcomes.

### Example 3: Resilience Training Pipeline

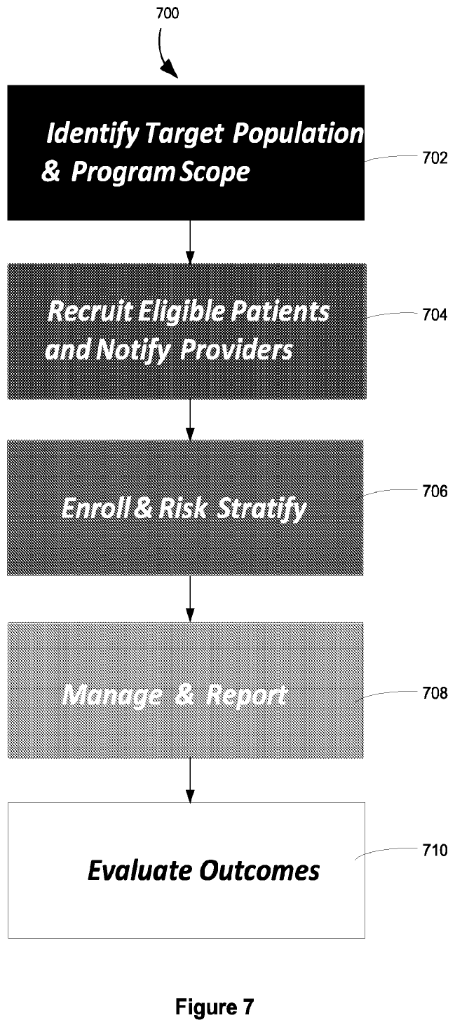

In FIG. 7, a method 700 for identifying and treating patients for inflammatory bowel disease is provided. First, information for a plurality of patients (e.g., subjects) diagnosed with IBD is evaluated 702 to identify patients who are eligible for resilience training and to determine desired outcomes for patients. In some embodiments, patient information includes medical history (e.g., disease severity) for each patient in the plurality of patients. In some embodiments, desired outcomes include coordinated care plan timeline, IBD improvement measures (e.g., decrease in inflammation) and/or number of medical procedures or adverse events (e.g., predetermined thresholds for minimizing medical procedures or adverse events). Eligible patients are then recruited (704) for resilience training (e.g., a first subset of eligible patients is obtained from the plurality of patients). Each patient in a second subset of patients (e.g., patients who agree to participate in resilience training) is enrolled in respective resilience training. Each patient in the second subset of patients (e.g., where each patient has a corresponding resilience score) is stratified (706) into a respective category in the plurality of categories (e.g., as described above with regards to blocks 220 and 222) based on respective resilience scores and disease severity (e.g., indicated by IBD severity score that is determined, in some embodiments, from medical history or other patient information), where each category in the plurality of categories has corresponding time-limited care plans. Based on assigned category, each patient in the second subset of patients participates in a respective time-limited care plan (e.g., patient care is managed 708). Patient outcomes are evaluated (710) for each patient in the second plurality of patients (e.g., annual outcome measurements are evaluated to determine if time-limited care plans are effective at improving patient health). In some embodiments, for each patient in the second subset of patients, if the respective time-limited care plan fails to meet a predefined outcome threshold (e.g., decrease in adverse events, etc.), the respective patient is assigned an alternative time-limited care plan (e.g., assigned to a different category associated with a different time-limited care plan in the plurality of categories or assigned to a different time-limited care plan within the initial respective category. In some embodiments, the effectiveness of time-limited care plans for respective patients is evaluated after a predetermined time point (e.g., after 3 months, after 6 months, after 12 months, etc.).

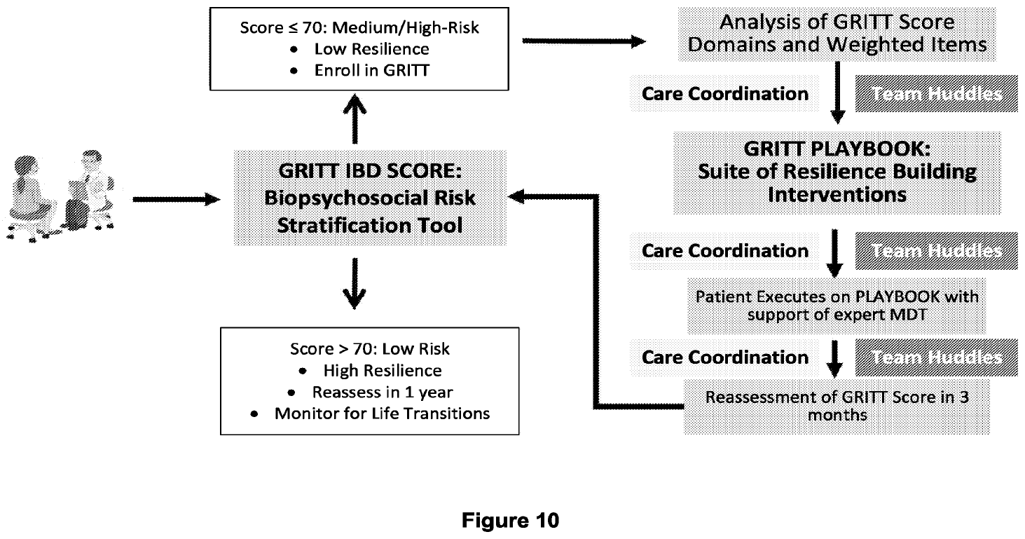

For example, FIG. 10 illustrates an example workflow of determining whether to enroll a patient in a care coordination program for resilience training and implementation of the care coordination program. In an embodiment, the care coordination program is the Gaining Resilience through Transitions (GRITT) Method.

GRITT Score.

The GRITT Score is a 22-item clinician administered assessment tool that captures the presence of disease interfering attitudes, beliefs or behaviors, collectively referred to as resilience, across 5 domains: general medical barriers affecting resilience, nutritional barriers affecting resilience, psychological barriers affecting resilience, lack of independent self-management skills affecting resilience and trouble with the healthcare system/access to care affecting resilience (see, for example, Keefer et al., “Validation of the Gaining Resilience Through Transitions [Gritt-IBD] Score: A Tool for Clinicians to Stratify Patients Based on Need for Team-Based Multidisciplinary Care,” Gastroenterology 2018; 154:S812-813, which is hereby incorporated herein by reference in its entirety). Higher scores are associated with less complexity, less disability, higher quality of life and well-being and higher resilience/psychological function. The GRITT score ranges from 0 (no resilience, severe biopsychosocial complexity) to 100 (high resilience, no biopsychosocial complexity), with a score of 70 being a cut-off for enrollment eligibility, in some implementations. The cut-off score for enrollment (e.g., 70) is based on risk of unplanned care (ED visits or hospitalizations), with ROC analyses yielding a 90% positive predictive value of unplanned care with a GRITT Score of 41 and a 90% negative predictive value of unplanned care at a GRITT Score of 69 (see, for example, Keefer et al., Gastroenterology 2018; 154:S812-813).

As illustrated in FIG. 10, in an embodiment, the resilience score is a GRITT score, and the determination to enlist a patient in the care coordination program (GRITT program) is based on whether the patient's GRITT score satisfies a care coordination threshold of 70. When the patient's GRITT score is 70 or lower, the patient is deemed to have low resilience and is enrolled in the GRITT program. When the patient's GRITT score is higher than 70, the patient is deemed to have high resilience, is not enrolled in the GRITT program, and undergoes monitoring for life transitions and reassessment after an elapsed time period (e.g., 1 year).

In some embodiments, enrollment in a care coordination program (e.g., the GRITT program) comprises resilience training via GRITT intervention.

GRITT Intervention.

The GRITT Method, short for Gaining Resilience through Transitions, is a resilience-based treatment methodology (see, for instance, Keefer et al., “Gaining Resilience Through Transitions in IBD [Gritt™-IBD], a Subspecialty Outpatient Medical Home is Feasible, Acceptable and Associated with Positive Outcomes in Year 1,” Gastroenterology 2018; 154; and Keefer et al., “A Resilience-based Care Coordination Program has a positive and durable impact on health care utilization in Inflammatory Bowel Diseases (IBD),” American Journal of Gastroenterology 2020; 115; each of which is hereby incorporated herein by reference in its entirety). The GRITT method aims to build upon a patient's personal strengths in order to achieve mental, emotional and physical well-being in the setting of a chronic condition. The method focuses on early identification and effective remediation of personal or health system barriers that interfere with a patient's ability to overcome the full range of obstacles that present over time when living with a chronic condition. The method is based at least in part on the principles of positive psychology and social-cognitive theory of health behavior change.

Personalized, integrated care plans use a treat-to-target and tight control approach to self-management. In other words, as further illustrated in FIG. 10, targets are chosen based on strengths and limitations identified in the baseline GRITT Score (e.g., analysis of GRITT score domains and weighted items). There are 5 “treat to target” resilience goals that the intervention team works with the patient on during the course of the program: 1) Disease acceptance; 2) Realistic optimism; 3) Social Support; 4) Self-confidence/self-efficacy; and 5) Self-regulatory skills. These targets are transdiagnostic and cut across specializations (e.g., nutrition targeting self-efficacy may focus on healthier nutrition choices, social work targeting self-efficacy may focus on bathroom anxiety and pharmacy targeting self-efficacy may focus on self-injection). For example, as illustrated in FIG. 10, resilience goals can be targeted by one or more interventions from a plurality of possible interventions (e.g., GRITT Playbook: Suite of Resilience Building Interventions). Targets are chosen based on strengths and limitations identified in the baseline GRITT Score and reviewed and agreed upon by a multidisciplinary care team (MCT), led by a health psychologist, in weekly huddles.

A patient is eligible for graduation when the MCT agrees that all targets have been optimized. As further illustrated in FIG. 10, efficacy of interventions can be reassessed after a period of execution has elapsed (e.g., 1 months, 3 months, 6 months, etc.), by calculating the patient's GRITT score, and determining whether the patient is eligible for graduation or whether a continued course is recommended.

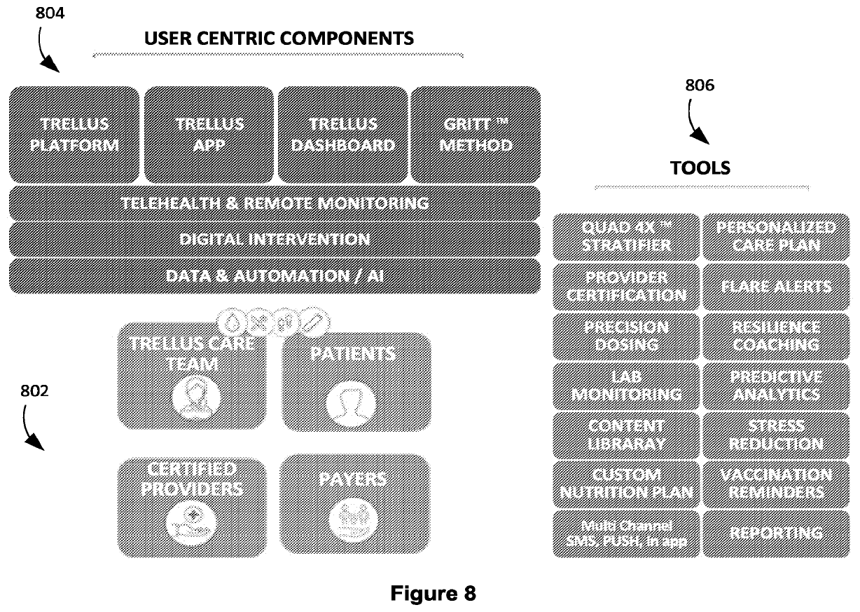

FIG. 8 illustrates multiple factors involved in providing excellent care to patients. This can be a complicated process, involving multiple components. For example, the determination of appropriate time-limited care plans requires the participation of multiple groups 802 (e.g., cooperation between patients, care teams, and health care providers). However, there are multiple components 804 available that enable patients to remotely interact with health care providers, receive care plan information, and/or participate in treatments (e.g., to receive resilience training or as part of time-limited care plans). In some embodiments, one or more of these remote access options corresponds to the input/output interfaces 106, as described with regards to FIG. 1. In addition, there is a plurality of care plan options (e.g., tools 806) that can be offered to patients to provide individualized time-limited care plans. By engaging all of the stakeholders 802, using appropriate communication and analysis options 804, and tailoring care plans to individual patients 906, the methods described herein serve to improve patient outcomes.

### REFERENCES

### References

1. Kaplan G G, Ng S C. Understanding and Preventing the Global Increase of Inflammatory Bowel Disease. Gastroenterology 2017; 152:313-321 e2.
2. Ng S C, Shi H Y, Hamidi N, et al. Worldwide incidence and prevalence of inflammatory bowel disease in the 21st century: a systematic review of population-based studies. Lancet 2017; 390:2769-2778.
3. Park K T, Ehrlich O G, Allen J I, et al. The Cost of Inflammatory Bowel Disease: An Initiative From the Crohn's & Colitis Foundation. Inflamm Bowel Dis 2020; 26:1-10.
4. Regueiro M, Click B, Anderson A, et al. Reduced Unplanned Care and Disease Activity and Increased Quality of Life After Patient Enrollment in an Inflammatory Bowel Disease Medical Home. Clin Gastroenterol Hepatol 2018; 16:1777-1785.
5. Szigethy E, Murphy S M, Ehrlich O G, et al. Mental Health Costs of Inflammatory Bowel Diseases. Inflamm Bowel Dis 2021; 27:40-48.
6. Szigethy E M, Allen J I, Reiss M, et al. White Paper AGA: The Impact of Mental and Psychosocial Factors on the Care of Patients With Inflammatory Bowel Disease. Clin Gastroenterol Hepatol 2017; 15:986-997.
7. Regueiro M. Medical Homes for Patients With Inflammatory Bowel Disease. Gastroenterol Hepatol (NY) 2017; 13:375-377.
8. Lores T, Goess C, Mikocka-Walus A, et al. Integrated Psychological Care Reduces Health Care Costs at a Hospital-Based Inflammatory Bowel Disease Service. Clin Gastroenterol Hepatol 2021; 19:96-103 e3.
9. Barberio B, Zamani M, Black C J, et al. Prevalence of symptoms of anxiety and depression in patients with inflammatory bowel disease: a systematic review and meta-analysis. Lancet Gastroenterol Hepatol 2021; 6:359-370.
10. Mikocka-Walus A, Knowles S R, Keefer L, et al. Controversies Revisited: A Systematic Review of the Comorbidity of Depression and Anxiety with Inflammatory Bowel Diseases. Inflamm Bowel Dis 2016; 22:752-62.
11. Keefer L. Editorial: depression in the setting of inflammatory bowel disease means we have failed to provide early, effective, psychosocial care. Aliment Pharmacol Ther 2017; 46:553-554.
12. Ludvigsson J F, Olen O, Larsson H, et al. Association between inflammatory bowel disease and psychiatric morbidity and suicide: A Swedish nationwide population-based cohort study with sibling comparisons. J Crohns Colitis 2021.
13. Keefer L, Kane S V. Considering the Bidirectional Pathways Between Depression and IBD: Recommendations for Comprehensive IBD Care. Gastroenterol Hepatol (N Y) 2017; 13:164-169.
14. Keefer L. What can we do to tackle anxiety and depression in patients with inflammatory bowel disease? Lancet Gastroenterol Hepatol 2021; 6:337-338.
15. Phillipou A, Sehgal P, Ungaro R C, Wang K, Bagiella E., Dubinsky M C, Keefer L. High Levels of Psychological Resilience Are Associated with Decreased Anxiety in Inflammatory Bowel Disease. Inflamm Bowel Dis 2021; in press.
16. Lenti M V, Cococcia S, Ghorayeb J, et al. Stigmatisation and resilience in inflammatory bowel disease. Intern Emerg Med 2020; 15:211-223.
17. van Erp L W, van Gerven J, Bloem S, et al. Acceptance and perceived control are independently associated with quality of life in inflammatory bowel disease: Introduction of a new segmentation model. J Crohns Colitis 2021.
18. Sirois F M, Hirsch J K. A longitudinal study of the profiles of psychological thriving, resilience, and loss in people with inflammatory bowel disease. Br J Health Psychol 2017; 22:920-939.
19. Chao C Y, Lemieux C, Restellini S, et al. Maladaptive coping, low self-efficacy and disease activity are associated with poorer patient-reported outcomes in inflammatory bowel disease. Saudi J Gastroenterol 2019; 25:159-166.
20. Keefer L, Kiebles J L, Taft T H. The role of self-efficacy in inflammatory bowel disease management: preliminary validation of a disease-specific measure. Inflamm Bowel Dis 2011; 17:614-20.
21. Kiebles J L, Doerfler B, Keefer L. Preliminary evidence supporting a framework of psychological adjustment to inflammatory bowel disease. Inflamm Bowel Dis 2010; 16:1685-95.
22. Sehgal P, Ungaro R C, Foltz C, et al. High Levels of Psychological Resilience Associated With Less Disease Activity, Better Quality of Life, and Fewer Surgeries in Inflammatory Bowel Disease. Inflamm Bowel Dis 2020.
23. Hirten R P, Danieletto M, Scheel R, et al. Longitudinal Autonomic Nervous System Measures Correlate With Stress and Ulcerative Colitis Disease Activity and Predict Flare. Inflamm Bowel Dis 2020.
24. Melinder C, Hiyoshi A, Fall K, et al. Stress resilience and the risk of inflammatory bowel disease: a cohort study of men living in Sweden. BMJ Open 2017; 7:e014315.
25. Chey W D, Keefer L, Whelan K, et al. Behavioral and Diet Therapies in Integrated Care for Patients With Irritable Bowel Syndrome. Gastroenterology 2021; 160:47-62.
26. Keefer L. Behavioural medicine and gastrointestinal disorders: the promise of positive psychology. Nat Rev Gastroenterol Hepatol 2018; 15:378-386.

### CONCLUSION

The terminology used herein is for the purpose of describing particular cases only and is not intended to be limiting. As used herein, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the terms “about” and “approximately” means within an acceptable error range for the particular value as determined by one of ordinary skill in the art, which depends in part on how the value is measured or determined, e.g., the limitations of the measurement system. For example, in some embodiments “about” mean within 1 or more than 1 standard deviation, per the practice in the art. In some embodiments, “about” means a range of ±20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. In some embodiments, the term “about” refers to ±10%. In some embodiments, the term “about” refers to ±5%.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A method of evaluating a subject for management of an inflammatory bowel disease (IBD), the method comprising:
determining a resilience score, within a range of resilience scores, for the subject, wherein each of a plurality of different resilience domain assessments of the subject contribute to the resilience score; and
using the resilience score to determine whether to enlist the subject in an IBD care coordination program, wherein:
when the resilience score fails to satisfy a care coordination threshold, the subject is not enlisted in the IBD care coordination program, and
when the resilience score satisfies the care coordination threshold, the method further comprises performing the IBD care coordination program by a procedure comprising:
determining an IBD severity score, within a range of IBD severity scores, for the subject based, at least in part, on a risk for one or more IBD complications or surgery to alleviate IBD;
using both the resilience score and the IBD severity score to assign the subject to a category in a plurality of categories, wherein each category in the plurality of categories is associated with a unique combination of a sub-range of the range of resilience scores and a sub-range of the range of IBD severity scores;
determining at least one time-limited care plan, at a first time point, for the subject based upon the identity of the assigned category within the plurality of categories; and
prioritizing the at least one time-limited care plan by an outcome of one or more resilience domain assessments in the plurality of different resilience domain assessments.

2. The method of claim 1, where:
the plurality of categories consists of a first category, a second category, a third category, and a fourth category, wherein:
the first category comprises a combination of a low resilience score and a high IBD severity score, and is associated with a care plan comprising (i) enrollment in a resilience program, (ii) close disease monitoring of the subject, (iii) tracking one or more of remote symptoms, subject reported outcomes, and biomarkers of the subject associated with IBD, (iv) application of one or more digital behavioral health maintenance and prevention tools associated with IBD, and (v) educational content customized to the subject;
the second category comprises a combination of a high resilience score and a high IBD severity score, and is associated with a care plan comprising (i) periodic resilience score reassessment, (ii) close disease monitoring of the subject, (iii) tracking one or more of remote symptoms, subject reported outcomes, and biomarkers of the subject associated with IBD, (iv) application of one or more digital behavioral health maintenance and prevention tools associated with IBD, and (v) featured educational content;
the third category comprises a combination of a low resilience score and a low IBD severity score, and is associated with a care plan comprising (i) enrollment in a resilience program, (ii) tracking one or more of remote symptoms, subject reported outcomes, and biomarkers of the subject associated with IBD, (iii) application of one or more digital behavioral health maintenance and prevention tools associated with IBD, and (iv) educational content customized to the subject; and
the fourth category comprises a combination of a high resilience score and a low IBD severity score and is associated with a care plan comprising (i) periodic resilience score reassessment, (ii) tracking one or more of remote symptoms, subject reported outcomes, and biomarkers of the subject associated with IBD, (iii) application of one or more digital behavioral health maintenance and prevention tools associated with IBD, and (iv) featured educational content.

3. The method of claim 1, wherein the plurality of different resilience domain assessments of the subject comprises two or more, three or more, four or more, or all five of:
(i) a general medical factor of the subject affecting resilience,
(ii) a measure of independence exhibited by the subject,
(iii) a nutritional and staminal assessment of the subject,
(iv) a psychosocial assessment of the subject, and
(v) an assessment of the present ability of the subject to access medical care.

4. The method of claim 3, wherein the plurality of different resilience domain assessments of the subject comprises a general medical factor of the subject, is one or more of:
(i) when the subject is in remission, a report of high impact of one or more physical symptoms on a plurality of daily functions,
(ii) when the subject has had at least one unplanned hospitalization or emergency department visit in the previous twelve months,
(iii) when the subject has had an IBD-related surgery in the previous six months,
(iv) when the subject has surgery planned within the next six months, and
(v) a report of chronic pain by the subject.

5. The method of claim 1, wherein the plurality of different resilience domain assessments of the subject comprises the subject's experience of physical health in the form of chronic pain, fatigue, urgency to use the bathroom, incontinence, nausea and recent or future health care use.

6. The method of claim 3, wherein the plurality of different resilience domain assessments of the subject comprises a measure of independence exhibited by the subject, and the subject is deemed to have inadequate independence when the subject is afraid to self-inject or undergo infusion or a necessary medical procedure, has a poor relationship with a caregiver or care team, is a child or young adult, has a poor attendance record at work or school, or has frequently cancelled or failed to attend medical appointments.

7. The method of claim 1, wherein:
the plurality of different resilience domain assessments of the subject comprises a measure of independence exhibited by the subject that indicates that the subject has inadequate independence; and
the one time-limited care plan comprises arranging to have the subject meet with a social worker and/or a clinical pharmacist associated with the one time-limited care plan.

8. The method of claim 1, wherein the at least one time-limited care plan comprises:
a cognitive behavioral therapy;
a low fermentable oligosaccharides, disaccharides, monosaccharides and polyols (FODMAP) diet; or
a self-directed hypnosis.

9. The method of claim 1, wherein the determining a resilience score is performed by having the subject complete a questionnaire in an application on a smart phone associated with the subject.

10. The method of claim 1, wherein the procedure further comprises repeating the determining the resilience score after a first predetermined period has elapsed since the first time point, wherein:
when the resilience score fails to satisfy the care coordination threshold, the subject is removed from the IBD care coordination program;
when the resilience score fails to satisfy the care coordination threshold and the subject exhibits indications of low resilience, the subject is reassessed to determine an updated resilience score after a second predetermined period has elapsed; and
when the resilience score continues to satisfy the care coordination threshold, the IBD care coordination program is continued.

11. The method of claim 10, wherein the predetermined period is at least a month, at least two months, at least three months, at least four months, at least six months, or between four months and eight months.

12. The method of claim 1, wherein the inflammatory bowel disease is ulcerative colitis or Crohn's disease.

13. The method of claim 1, wherein the inflammatory bowel disease is Crohn's disease and the IBD severity score is determined based on one or more criteria selected from the group consisting of:
the AGA risk stratification tool;
presence or absence of current or historical perianal disease;
presence or absence of moderate to severe rectal disease;
presence or absence of extensive disease including ileal involvement greater than 40 cm or pancolitis;
presence or absence of large or deep mucosal lesions;
history of IBD related hospitalization within last 12 months;
presence of stoma;
prior intestinal resections;
presence or absence of stricturing disease;
presence of bowel fistulas (internal penetrating);
steroid dependent disease or use within last 12 months;
current or historical use of biologics, small molecules or immunomodulators; and
lack of symptomatic improvement with prior exposure to biologics and/or immunomodulators.

14. The method of claim 2, wherein:
the inflammatory bowel disease is Crohn's disease;
the IBD severity score is determined based on one or more criteria selected from the group consisting of:
the AGA risk stratification tool;
presence or absence of current or historical perianal disease;
presence or absence of moderate to severe rectal disease;
presence or absence of extensive disease including ileal involvement greater than 40 cm or pancolitis;
presence or absence of large or deep mucosal lesions;
history of IBD related hospitalization within last 12 months;
presence of stoma;
prior intestinal resections;
presence or absence of stricturing disease;
presence of bowel fistulas (internal penetrating);
steroid dependent disease or use within last 12 months;
current or historical use of biologics, small molecules or immunomodulators; and
lack of symptomatic improvement with prior exposure to biologics and/or immunomodulators; and
the IBD severity score is high-risk.

15. The method of claim 1, wherein the inflammatory bowel disease is ulcerative colitis and the IBD severity score is based on one or more criteria selected from the group consisting of:
the AGA risk stratification tool;
presence or absence of deep ulcers on the subject's latest colonoscopy;
history of IBD related hospitalization within last 12 months;
steroid dependent disease or use within last 12 months;
current or historical use of biologics, small molecules or immunomodulators; and
lack of symptomatic improvement with prior exposure to biologics, small molecules and/or immunomodulators.

16. The method of claim 2, wherein:
the inflammatory bowel disease is ulcerative colitis;
the IBD severity score is determined based on one or more criteria selected from the group consisting of:
the AGA risk stratification tool;
presence or absence of deep ulcers on the subject's latest colonoscopy;
history of IBD related hospitalization within last 12 months;
steroid dependent disease or use within last 12 months;
current or historical use of biologics, small molecules or immunomodulators; and
lack of symptomatic improvement with prior exposure to biologics, small molecules and/or immunomodulators; and
the IBD severity score is high-risk.

17. The method of claim 1, wherein the at least one time-limited care plan includes managing IBD medication for the subject, managing IBD related appointment keeping for the subject, tracking one or more IBD related health issues for the subject, or managing daily activities for the subject.

18. A non-transitory computer readable storage medium for evaluating a subject for management of an inflammatory bowel disease (IBD), wherein the non-transitory computer readable storage medium stores instructions, which when executed by a computer system, cause the computer system to:
determine a resilience score, within a range of resilience scores, for the subject, wherein each of a plurality of different resilience domain assessments of the subject contribute to the resilience score; and
use the resilience score to determine whether to enlist the subject in an IBD care coordination program, wherein:
when the resilience score fails to satisfy a care coordination threshold, the subject is not enlisted in the IBD care coordination program,
when the resilience score satisfies the care coordination threshold, the instructions further comprise performing the IBD care coordination program by a procedure comprising:
determining an IBD severity score, within a range of IBD severity scores, for the subject based, at least in part, on a risk for one or more IBD complications or surgery to alleviate IBD;
using both the resilience score and the IBD severity score to assign the subject into a category in a plurality of categories, wherein each category in the plurality of categories is associated with a unique combination of a sub-range of the range of resilience scores and a sub-range of the range of IBD severity scores;
determining at least one time-limited care plan, at a first time point, for the subject based upon the identity of the assigned category within the plurality of categories; and
prioritizing the at least one time-limited care plan by an outcome of one or more resilience domain assessments in the plurality of different resilience domain assessments.

19. A computer system for evaluating a subject for management of an inflammatory bowel disease (IBD), comprising:
one or more processors;
memory; and
one or more programs stored in the memory for execution by the one or more processors, the one or more programs comprising instructions for:
determining a resilience score, within a range of resilience scores, for the subject, wherein each of a plurality of different resilience domain assessments of the subject contribute to the resilience score;
using the resilience score to determine whether to enlist the subject in an IBD care coordination program, wherein:
when the resilience score fails to satisfy a care coordination threshold, the subject is not enlisted in the IBD care coordination program, and
when the resilience score satisfies the care coordination threshold, the method further comprises performing the IBD care coordination program by a procedure comprising:
determining an IBD severity score, within a range of IBD severity scores, for the subject based, at least in part, on a risk for one or more IBD complications or surgery to alleviate IBD;
using both the resilience score and the IBD severity score to assign the subject to a category in a plurality of categories, wherein each category in the plurality of categories is associated with a unique combination of a sub-range of the range of resilience scores and a sub-range of the range of IBD severity scores;
determining at least one time-limited care plan, at a first time point, for the subject based upon the identity of the assigned category within the plurality of categories; and
prioritizing the time-limited care plan by an outcome of one or more resilience domain assessments in the plurality of different resilience domain assessments.
